(19) United States
(12) Patent Application Publication
(10) Pub. No.: US 2026/0257796 A1
(43) Pub. Date: Sep. 3, 2026

(54) Aircraft Cabin Medical Telemetry System With Governed Contextual Memory and Readiness-Conditioned Reliance

(71) Applicant: OneSource Solutions International, Inc., Sudbury, MA (US)

(72) Inventors: Harold Arkoff, Sudbury, MA (US); Vedran Jukic, Trieste (IT)

(73) Assignee: OneSource Solutions International, Inc., Sudbury, MA (US)

(21) Appl. No.: 19/669,881

(22) Filed: May 6, 2026

Related U.S. Application Data
(63) Continuation-in-part of application No. 18/731,145, filed on May 31, 2024, now Pat. No. 12,665,083.

(51) Int. Cl. B64D 11/00; G16H 10/60; H04L 67/12

(52) U.S. Cl. CPC B64D 11/00; G16H 10/60; H04L 67/12

(57) ABSTRACT

An aircraft cabin medical telemetry system receives telemetry from wearable or medical devices through proxy-capable cabin nodes. The system associates the telemetry with passenger identity, seat, device, flight, consent, timing, and provenance context, and stores the telemetry or derived values as memory atoms in patient-partitioned contextual memory. A retrieval engine selects candidate memory atoms for a monitoring task. Before execution, a non-bypassable memory gate evaluates the candidate memory atoms under an aviation policy snapshot and integrity criteria. Admitted records are assembled into a governed context bundle. A constrained execution interface permits a monitoring or reasoning component to execute only using the governed context bundle and an execution governance artifact. Downstream reliance, including crew notification, documentation, display, or off-aircraft transmission to a remote monitoring center, is conditioned on verification of a readiness artifact. The system may operate as a cabin medical overlay isolated from flight-control systems.

### CROSS-REFERENCE TO RELATED APPLICATIONS

This application is a continuation-in-part of U.S. Patent Application No. 18/731,145, filed May 31, 2024, which is a divisional of U.S. Patent Application Ser. No. 18/518,945, filed Nov. 24, 2023, now U.S. Pat. No. 12,002,579.

The disclosures of the foregoing applications are incorporated by reference in their entirety. In the event of any inconsistency between the present disclosure and an application incorporated by reference, the present disclosure governs.

### FIELD OF THE INVENTION

The present disclosure relates to aircraft cabin medical telemetry systems and to controlled processing of medical data acquired during flight. More particularly, the disclosure relates to systems and methods for receiving telemetry from wearable or medical devices through aircraft cabin nodes, associating the telemetry with a passenger identity, storing the telemetry in patient-partitioned contextual memory, evaluating the stored telemetry under aviation-specific policy controls, and conditioning monitoring, transmission, display, documentation, or other downstream reliance on verified governance and readiness artifacts.

The disclosure also relates to retrofit aircraft cabin medical overlay networks that operate independently of flight-control systems and primary avionics buses while supporting medical telemetry capture, proxy node handoff, governed context assembly, constrained execution of monitoring or reasoning components, controlled off-aircraft transmission, remote monitoring verification, audit logging, and fail-closed or fail-quiet operation.

### BACKGROUND OF THE INVENTION

Aircraft cabins are not normally equipped as controlled medical telemetry environments. During an in-flight medical event, cabin crew typically rely on passenger self-reporting, visual observation, onboard emergency medical kits, available passenger clinicians, crew procedures, and voice communication with a ground-based medical support service. These procedures may be sufficient for many events, but they do not ordinarily provide continuous patient-specific telemetry, reliable device-to-passenger binding, governed use of passenger-worn device data, deterministic provenance, or controlled transmission of medical data to a remote monitoring center.

Passengers may carry wearable devices capable of measuring heart rate, oxygen saturation, rhythm-related information, activity state, temperature, glucose-related information, or other physiologic indicators. Cabin crew may also deploy portable medical equipment during an emergency. However, the presence of such devices does not by itself create a reliable in-flight monitoring system. Device data may be incomplete, stale, unauthenticated, associated with the wrong person, associated with an uncertain seat location, produced under unknown consent conditions, or unsuitable for clinical reliance without additional verification.

Aircraft cabins also present communication and integration constraints. A cabin may include passenger electronic devices, seat electronics, in-flight entertainment equipment, wireless access points, satellite connectivity equipment, crew mobile devices, service carts, portable emergency equipment, and operator-specific procedures. A medical telemetry system deployed in this environment should not require modification of flight-control systems, connection to primary avionics buses, interference with aircraft communications, or replacement of existing cabin systems. It should also tolerate intermittent connectivity, passenger movement, seat changes, battery limitations, radio interference, and loss of off-aircraft communication.

Conventional remote medical support workflows are also limited by the quality and structure of information available to the remote service. A ground-based clinician may receive verbal descriptions from crew, isolated measurements, or delayed updates. The remote clinician may not receive a governed context showing source identity, device identity, passenger identity, seat association, timing, consent state, confidence, transformation history, or policy status for the data being discussed. As a result, the remote support service may be asked to rely on information whose provenance and admissibility are uncertain.

Existing aircraft communication systems are generally designed for passenger connectivity, crew communication, entertainment, operational messaging, or aircraft services. They are not normally configured to create patient-partitioned medical memory, evaluate captured telemetry under aviation-specific medical-data policies, prevent monitoring or reasoning components from using unadmitted patient-scoped context, or require a machine-verifiable readiness artifact before downstream reliance occurs.

Similarly, generic medical telemetry systems do not address the particular requirements of an aircraft cabin. A hospital bedside telemetry system may assume a fixed bed, a known patient encounter, installed medical devices, local network infrastructure, and established clinical information systems. An aircraft cabin may instead involve temporary patient identity resolution, passenger-carried devices, crew-carried devices, seat-associated proxy nodes, portable dongles, intermittent satellite links, route-dependent privacy conditions, limited crew workflow, and strict non-interference with aircraft systems.

Software-based monitoring and reasoning tools introduce additional risks if they can access telemetry directly or if their outputs are automatically transmitted, displayed, documented, or relied upon without controlled admission of the underlying context. A monitoring component may generate a plausible alert or recommendation from stale, conflicting, unauthenticated, or incorrectly associated telemetry. In an aircraft cabin, such an output may affect crew attention, remote medical consultation, documentation, diversion discussions, or other operational decisions. Therefore, the system should control not only capture of data, but also whether data is admitted for execution and whether outputs may be used in a downstream reliance event.

There is a need for an aircraft-cabin medical telemetry architecture that can be deployed as a retrofit or overlay system, receive telemetry from wearable and medical devices through proxy-capable cabin nodes, associate the telemetry with passenger, seat, device, flight, consent, and time context, store the telemetry in patient-partitioned contextual memory, evaluate candidate records under an aviation policy snapshot, assemble admitted records into a governed context bundle, constrain execution of monitoring or reasoning components to admitted context, and condition transmission, display, documentation, crew notification, remote monitoring, or other downstream reliance on verification of a readiness artifact.

There is also a need for such an architecture to preserve crew authority and aircraft safety by operating independently of flight-control systems and primary avionics buses, avoiding autonomous aircraft control, controlling off-aircraft egress of medical data, maintaining audit records, supporting proxy handoff as a passenger or device moves, supporting multiple passenger partitions, adapting to cabin radio conditions, and failing closed or quiet when governance, readiness, connectivity, or device-authentication conditions are not satisfied.

### SUMMARY OF THE INVENTION

The present disclosure describes an aircraft-cabin medical telemetry architecture that receives medical or physiologic telemetry during flight, associates the telemetry with a passenger identity, stores the telemetry in patient-partitioned contextual memory, controls use of the telemetry by monitoring or reasoning components, and conditions downstream reliance on verification of machine-readable governance artifacts.

In some embodiments, the architecture includes a retrofit clinical integration plane deployed in or used within an aircraft cabin. The clinical integration plane may include proxy-capable cabin nodes located at or associated with seat positions, crew-carried devices, passenger-carried devices, portable dongles, medical device adapters, cabin gateways, carts, emergency medical kits, or other cabin components. The proxy-capable cabin nodes receive telemetry from wearable devices, passenger-carried devices, and medical devices deployed during an in-flight medical event.

The architecture associates received telemetry with context information. The context information may include passenger identity, seat assignment, flight identifier, device identifier, proxy node identifier, cabin location, consent state, time information, route or jurisdiction information, source trust information, and communication state. Passenger identity may be resolved using one or more sources, including manifest information, seat assignment, device association, passenger credential, consent state, emergency token, temporary patient profile, crew verification, companion confirmation, remote clinician confirmation, or post-event reconciliation.

The received telemetry and related derived values are stored as memory atoms in a patient-partitioned contextual memory. Each memory atom may include the telemetry or derived value together with provenance metadata, timing metadata, device metadata, identity metadata, consent metadata, trust metadata, and transformation metadata. The patient-partitioned contextual memory maintains separation between passengers and may maintain separate flight-session memory regions for multiple passengers in the same aircraft cabin.

When a monitoring task is requested, a retrieval process selects candidate memory atoms from the patient-partitioned contextual memory. Before a monitoring or reasoning component is permitted to use the candidate memory atoms, a non-bypassable memory gate evaluates the candidate memory atoms under an aviation policy snapshot and applicable integrity criteria. The aviation policy snapshot may encode airline operator rules, route-dependent or jurisdiction-dependent requirements, consent rules, credentialing rules, escalation thresholds, retention rules, crew role permissions, remote clinician permissions, connectivity constraints, and emergency-mode rules.

The non-bypassable memory gate determines whether each candidate memory atom is admitted, denied, redacted, abstracted, attenuated, quarantined, or transformed into a non-data-bearing form. Records that are not admitted or transformed under the aviation policy snapshot are not made available as patient-scoped input to the monitoring or reasoning component. The admitted memory atoms are assembled in a deterministic manner into a governed context bundle.

A constrained execution interface permits the monitoring or reasoning component to execute only when a governed execution package is available. The governed execution package includes the governed context bundle and an execution governance artifact. If the governed context bundle or execution governance artifact is absent, invalid, expired, mismatched, or inconsistent with the aviation policy snapshot, execution may be denied, delayed, quarantined, or limited to non-patient-specific fallback content.

Outputs from the monitoring or reasoning component do not automatically cause operational reliance. A downstream reliance event is conditioned on verification of a readiness artifact bound to the governed context bundle and the aviation policy snapshot. The readiness artifact may include a bundle identifier, policy snapshot identifier, timestamp, digest, signature, nonce, evaluator identifier, execution certificate, readiness certificate, policy-verification result, or transmission authorization record. Downstream reliance events may include crew notification, display, rendering, storage as actionable medical information, documentation, physician coordination, transmission to a remote monitoring center, remote monitoring center acceptance, forwarding, or diversion-related decision support.

In some embodiments, a governed connector controls off-aircraft transmission. The governed connector may permit satellite, air-to-ground, Wi-Fi, cellular, or other off-aircraft transmission of telemetry, alerts, recommendations, or summaries only after readiness verification. A remote monitoring center may independently verify the readiness artifact before accepting, storing, displaying, forwarding, documenting, or acting upon a received payload. If readiness verification fails, the payload may be rejected, quarantined, held for retransmission, recorded as a failed-readiness event, or prevented from being presented as actionable medical information.

The architecture may generate audit records that preserve the path from telemetry capture to reliance. Such records may include device identifiers, proxy node identifiers, passenger identity references, seat identifiers, flight identifiers, memory atom identifiers, admissibility results, aviation policy snapshot identifiers, governed context bundle identifiers, execution governance artifacts, readiness artifacts, transmission records, receipt records, remote verification outcomes, proxy handoff records, and failed-readiness records.

In some embodiments, the architecture supports proxy handoff. When a passenger moves, a device moves, a node becomes unavailable, link quality degrades, connectivity changes, or a different cabin node becomes a better proxy, the system may select a new proxy-capable cabin node while preserving provenance continuity between the prior proxy node and the newly selected proxy node.

The architecture may operate as a cabin medical overlay network. In some embodiments, the overlay is logically isolated from flight-control systems and primary avionics buses. The architecture does not command aircraft navigation, propulsion, flight-control, or primary avionics functions and does not autonomously divert an aircraft or administer treatment. Crew authority and existing emergency procedures are preserved.

The architecture may also adapt to aircraft cabin radio and connectivity constraints. It may adjust transmit power, duty cycle, scanning interval, channel selection, retry behavior, buffering behavior, proxy selection, or fail-quiet operation in response to degraded wireless performance, detected interference, satellite connectivity loss, or other cabin radio coexistence constraints.

The disclosed architecture therefore provides a controlled technical path for aircraft-cabin medical telemetry. It controls capture, identity association, patient partitioning, policy-based admission, governed context assembly, constrained execution, readiness-conditioned reliance, off-aircraft transmission, remote verification, and auditability in an aircraft cabin environment.

### BRIEF DESCRIPTION OF THE DRAWINGS

FIG. 1 is a schematic diagram of an aircraft cabin medical telemetry system including cabin telemetry capture, patient-partitioned contextual memory, governed execution, readiness-conditioned reliance, remote monitoring, audit logging, and isolation from aircraft operational systems.

FIG. 2 is a schematic diagram of proxy-capable cabin nodes configured to receive telemetry from wearable or medical devices and to route the telemetry through selected proxy paths, including proxy handoff and buffering.

FIG. 3 is a schematic diagram of passenger identity resolution and patient-partitioned contextual memory, including identity inputs, resolved or temporary passenger identity states, memory partitions, and memory atoms.

FIG. 4 is a flow diagram showing selection of candidate memory atoms, policy-based gate evaluation, governed context bundle assembly, and constrained execution of a monitoring or reasoning component.

FIG. 5 is a flow diagram showing readiness artifact verification before downstream reliance, including local reliance, off-aircraft transmission, receiver-side verification, remote monitoring, quarantine, and receipt-bound audit records.

FIG. 6 is a schematic diagram showing aircraft cabin network isolation, RF coexistence control, mediated egress, buffering, fail-closed operation, fail-quiet operation, and fallback to standard cabin medical procedures.

### DETAILED DESCRIPTION OF THE INVENTION

### Definitions

The following definitions are provided to clarify selected terminology used in the present disclosure. The definitions are illustrative and are not intended to exclude equivalent structures, functions, or implementations unless expressly stated. A term may be used in singular or plural form.

The term “aircraft cabin” refers to a passenger-carrying or crew-accessible environment of an aircraft, including a commercial aircraft, private aircraft, charter aircraft, air ambulance, military aircraft, cargo aircraft configured for passenger transport, or other aircraft environment in which a passenger, crew member, patient, or other person may experience a medical event during flight. An aircraft cabin may include passenger seating, crew areas, galleys, lavatories, aisles, service areas, seat electronics, cabin wireless equipment, emergency medical kits, crew devices, passenger devices, or cabin connectivity systems.

In this disclosure, “aircraft-cabin medical telemetry” means telemetry, physiologic data, device data, derived values, device status information, communication state information, or related medical-event information received, generated, routed, processed, or stored in connection with a person located in an aircraft cabin.

The term “cabin medical overlay network” denotes a physical, logical, software-defined, portable, retrofit, or hybrid communication and processing environment configured to support medical telemetry capture, routing, governance, execution, transmission, audit, or reliance within or in association with an aircraft cabin. A cabin medical overlay network may operate separately from passenger entertainment systems, passenger Internet systems, flight-control systems, and primary avionics buses.

For purposes of this disclosure, “retrofit clinical integration plane” refers to a controlled acquisition and routing layer deployed within or associated with an aircraft cabin to receive telemetry from wearable devices, portable medical devices, medical-grade devices, proxy-capable aircraft cabin nodes, dongles, adapters, crew-carried devices, passenger-carried devices, or cabin gateways and to provide such telemetry to downstream governance components. A retrofit clinical integration plane may be installed, carried, temporarily activated, or software-enabled without requiring structural redesign of the aircraft cabin.

The expression “proxy-capable aircraft cabin node” identifies a cabin-available component, device, adapter, dongle, gateway, mobile device, seat-associated endpoint, crew-carried device, passenger-carried device, cart-mounted device, battery-powered node, wall-powered node, seat-powered node, or temporarily active emergency node configured to receive, route, buffer, authenticate, normalize, or forward telemetry from a wearable device or medical device.

An “active proxy node” is a proxy-capable aircraft cabin node selected to receive or route telemetry for a particular telemetry source, passenger, temporary patient profile, device, seat location, cabin location, or medical event.

A “candidate proxy node” is a proxy-capable aircraft cabin node that is available or potentially available to serve as an active proxy node.

The phrase “proxy handoff” refers to a change from one active proxy node to another active proxy node for a telemetry source, passenger, temporary patient profile, or medical event. Proxy handoff may occur in response to passenger movement, device movement, node unavailability, degraded link quality, authentication state, battery state, cabin-location change, crew intervention, medical-device deployment, or policy change.

The phrase “provenance continuity” means preservation of the chain of source, device, proxy, gateway, communication path, timing, transformation, and handling information across telemetry capture, proxy selection, proxy handoff, memory atom creation, gate evaluation, execution, transmission, and audit.

The term “telemetry” includes measured, sensed, generated, transmitted, or derived information associated with a wearable device, portable medical device, medical-grade device, passenger device, crew device, dongle, adapter, or other source. Telemetry may include physiologic measurements, waveforms, trends, device status, alarm state, connection state, battery state, communication state, signal quality, source time, event time, sequence information, or derived values.

A “normalized telemetry object” is a machine-readable record generated from raw or device-specific telemetry and having standardized fields, such as device identity, device type, proxy node identity, passenger or seat association, cabin location, flight identifier, event time, receipt time, measurement value, units, data quality, communication state, source trust state, or provenance.

The phrase “passenger identity resolution” refers to a process of associating telemetry, a device, a temporary patient profile, or a medical event with a passenger identity or patient scope. Passenger identity resolution may use passenger manifest data, seat assignment data, boarding records, travel records, device association records, passenger mobile credentials, crew verification, companion or family confirmation, remote clinician confirmation, emergency tokens, biometric verification where permitted, temporary patient profiles, or post-event reconciliation.

A “resolved passenger identity” is a passenger identity determined or associated by the system using one or more identity inputs. A resolved passenger identity may be confirmed, probable, temporary, reconciled, or otherwise confidence-rated.

The term “passenger identity confidence state” denotes a machine-readable state indicating the confidence or status of an identity association. The confidence state may indicate confirmed identity, probable identity, temporary identity, anonymous emergency partition, conflicting identity, pending reconciliation, or unresolved identity.

The term “temporary patient profile” refers to a patient-scoped profile created during an in-flight medical event when a final passenger identity is unavailable, incomplete, uncertain, or pending. A temporary patient profile may include a temporary profile identifier, flight identifier, seat or cabin location, device association, proxy node association, crew observations, event time, consent state, emergency state, and available identity information.

An “anonymous emergency patient partition” is a patient-scoped memory partition used when passenger identity is not yet resolved or when policy requires temporary identity minimization. The partition may store telemetry and contextual information associated with an emergency event while limiting or omitting identifying passenger information until reconciliation is permitted.

An “emergency token” is a machine-readable identifier or control object used to associate telemetry, crew observations, device records, remote monitoring records, audit records, or other event information with a temporary patient profile or emergency patient partition before final identity resolution.

The phrase “post-event reconciliation” refers to a process performed during flight, after connectivity restoration, after landing, or during later review to confirm, correct, merge, separate, or maintain unresolved identity, consent, device association, telemetry, or audit records associated with an in-flight medical event.

The term “consent state” denotes a machine-readable representation of whether a passenger or authorized workflow has granted, denied, limited, revoked, not yet provided, or is subject to emergency-use handling for capture, processing, transmission, display, documentation, retention, remote monitoring, or other use of medical telemetry or patient-scoped information.

The term “patient-partitioned contextual memory” refers to a structured memory environment logically segmented by passenger identity, temporary patient profile, anonymous emergency patient partition, emergency token, flight-session region, or other patient-scoping identifier. Patient-partitioned contextual memory stores telemetry and related contextual information in a manner that maintains separation between passengers or patient profiles.

A “flight-session memory region” is a memory region associated with a flight, flight segment, passenger identity, temporary patient profile, emergency token, seat identifier, device identifier, or other flight-specific partitioning value. A flight-session memory region may persist for a flight, medical event, retention period, or other policy-defined interval.

A “memory atom” is a structured contextual record stored in patient-partitioned contextual memory. A memory atom may include telemetry, a derived value, device state, alarm state, communication state, crew observation, consent event, escalation event, remote clinician note, proxy handoff record, transmission record, readiness verification outcome, or other patient-scoped contextual information together with metadata for provenance, time validity, identity, device, proxy, consent, policy, transformation, or audit.

The phrase “provenance metadata” refers to metadata identifying the origin, source path, handling path, communication path, transformation path, device, proxy node, gateway, user, software component, remote service, or other source-related information associated with telemetry, memory atoms, governed context bundles, execution outputs, readiness artifacts, transmission payloads, or audit records.

The phrase “time-validity metadata” refers to metadata indicating source time, device time, event time, receipt time, processing time, validity interval, expiration time, sequence number, freshness state, stale-data state, supersession state, or other time-related information used to determine whether a record remains suitable for admission, execution, transmission, documentation, or reliance.

The term “source trust classification” means a classification indicating the trust, reliability, authority, or permitted use of a telemetry source or contextual record. Source trust classifications may distinguish medical-grade device data, wearable-device data, passenger-device data, crew-entered observations, remote clinician notes, derived values, buffered records, replayed records, unauthenticated records, or audit-only records.

The phrase “transformation history” refers to a record of normalization, unit conversion, redaction, abstraction, attenuation, quarantine, filtering, denial, admission, confidence adjustment, conversion into a non-data-bearing form, or other processing applied to telemetry, memory atoms, context bundles, payloads, or related records.

An “aviation policy snapshot” is a defined policy state applicable to telemetry capture, admission, transformation, execution, transmission, display, documentation, retention, remote monitoring, or downstream reliance in an aircraft-cabin environment. An aviation policy snapshot may encode airline or operator rules, route-dependent requirements, jurisdictional privacy rules, consent rules, credentialing constraints, escalation thresholds, retention rules, crew role permissions, remote clinician permissions, device trust rules, connectivity rules, emergency-mode rules, or flight-phase-dependent rules.

A “policy snapshot identifier or digest” is an identifier, version, hash, digest, signature, or other machine-readable reference associated with an aviation policy snapshot and usable to bind memory-gate decisions, governed context bundles, execution governance artifacts, readiness artifacts, transmission decisions, receipt-bound governance records, or audit records to the policy state used.

The term “monitoring task request” identifies a request or trigger for patient-specific monitoring, reasoning, alerting, summarization, documentation preparation, remote monitoring package creation, transmission preparation, or other task using patient-scoped contextual information. A monitoring task request may be initiated by crew, a cabin gateway, a rules engine, an AI-assisted module, a remote monitoring center, a device, or another authorized component.

A “candidate memory atom” is a memory atom selected or retrieved as potentially relevant to a monitoring task request before admission by the non-bypassable memory gate.

The term “retrieval engine” refers to a component, service, process, or logic configured to select candidate memory atoms from patient-partitioned contextual memory based on a monitoring task request, passenger identity, temporary patient profile, flight identifier, time window, device type, source trust state, escalation state, consent state, or other retrieval criteria.

A “non-bypassable memory gate” is a control function that prevents a monitoring or reasoning component from accessing patient-scoped contextual information unless candidate memory atoms have been evaluated under an applicable aviation policy snapshot and integrity criteria. A non-bypassable memory gate may be implemented as a policy enforcement point, validation controller, admission controller, access-control service, context firewall, execution precondition service, policy filter, trust broker, gateway service, edge service, or equivalent control mechanism.

The phrase “integrity criteria” refers to one or more checks applied before a memory atom is admitted or transformed for use. Integrity criteria may include provenance verification, temporal validity evaluation, source trust classification, conflict detection, completeness evaluation, device-authentication checking, proxy-node authentication checking, seat-identity consistency checking, consent-state checking, route-policy checking, jurisdiction-policy checking, communication-state checking, or transformation-history evaluation.

An “admissibility state” is a machine-readable result assigned to a candidate memory atom or candidate memory atom set by the non-bypassable memory gate. Admissibility states may include admission, denial, redaction, abstraction, attenuation, quarantine, or transformation into a non-data-bearing form.

“Redaction” means removal or masking of a portion of a record before use, transmission, display, documentation, or reliance.

“Abstraction” means replacement of a specific value, identifier, or record element with a less specific representation, such as a category, range, trend, summary, or non-identifying representation.

“Attenuation” means reduction in detail, precision, confidence, frequency, scope, or permitted downstream use of a record or output.

“Quarantine” refers to retention of a record, payload, output, or transmission in a state that prevents use as patient-scoped execution input or actionable downstream information unless later verification, review, policy update, or other required condition is satisfied.

A “non-data-bearing form” is a representation that preserves a structural, audit, policy, denial, or placeholder indication without exposing the underlying patient-scoped data.

An “admissibility result record” is a machine-readable record identifying the result of gate evaluation for a candidate memory atom or set of candidate memory atoms. The record may include a memory atom identifier, patient partition identifier, task identifier, aviation policy snapshot identifier, integrity criteria applied, admissibility state, transformation applied, denial reason, quarantine reason, confidence state, evaluator identifier, timestamp, or rule identifier.

A “governed context bundle” is a deterministically assembled set of admitted memory atoms and permitted transformed records prepared for execution by a monitoring or reasoning component. A governed context bundle may include admitted records, transformed records, passenger identity reference, flight identifier, task identifier, policy snapshot identifier, timestamp, transformation records, admissibility results, bundle identifier, or bundle digest.

A “governed context bundle assembler” is a component, service, process, or logic configured to assemble admitted memory atoms and permitted transformed records into a governed context bundle.

The phrase “canonical serialization” refers to a deterministic representation of a governed context bundle, readiness artifact, execution governance artifact, transmission payload, or other machine-readable object using defined ordering, field representation, encoding, timestamp formatting, measurement units, and treatment of missing or transformed values.

A “bundle identifier” is an identifier associated with a governed context bundle.

A “bundle digest” is a hash, digest, signature, checksum, or other integrity value computed over at least a portion of a governed context bundle or its canonical serialization.

An “execution governance artifact” is a machine-readable artifact associated with a governed context bundle and used to authorize or verify execution of a monitoring or reasoning component. The artifact may include bundle identifier, bundle digest, policy snapshot identifier, task identifier, passenger identity reference, flight identifier, admissibility result records, transformation records, execution authorization state, expiration time, evaluator identifier, component identifier, signature, or other verification data.

A “governed execution package” is a machine-readable package that includes a governed context bundle and an execution governance artifact, and that is required or checked before patient-specific execution by a monitoring or reasoning component.

The term “constrained execution interface” refers to an interface, service, controller, execution environment, gateway, or other mechanism that permits a monitoring or reasoning component to execute on patient-scoped information only when the required governed context bundle and execution governance artifact are present and valid.

The phrase “monitoring or reasoning component” refers to a rule engine, threshold detector, signal-processing module, trend module, predictive model, AI-assisted case manager, alert generator, summarization module, documentation-preparation module, remote monitoring package generator, or other software or hardware component configured to process admitted patient-scoped context and generate an advisory output, alert, recommendation, summary, trend, classification, documentation draft, or transmission payload.

The expression “non-patient-specific fallback content” identifies content that does not rely on patient-scoped contextual information and may include standard cabin medical procedure prompts, general emergency workflow guidance, crew checklist information, device troubleshooting guidance, or instructions to obtain additional verification.

A “readiness artifact” is a machine-verifiable control object that conditions downstream reliance on an output generated from a governed context bundle. A readiness artifact may be bound to the governed context bundle and aviation policy snapshot and may include a bundle identifier, policy snapshot identifier, time reference, verification digest, digital signature, nonce, evaluator identifier, execution certificate, readiness certificate, policy-verification result, transmission authorization record, or other verification data.

The phrase “readiness verification” refers to checking whether a readiness artifact is present, unexpired, valid, signed when required, bound to the correct governed context bundle, bound to the applicable aviation policy snapshot, consistent with the execution governance artifact, consistent with the task identifier, consistent with the passenger identity reference, and valid for the requested reliance type.

A “downstream reliance event” is an action or system state that relies upon an output generated from patient-scoped telemetry or a monitoring or reasoning component. Downstream reliance events may include crew notification, display, rendering, storage as actionable medical information, documentation, physician coordination, off-aircraft transmission, remote monitoring center acceptance, forwarding, or diversion-related decision support.

A “governed connector” is a controlled egress, transmission, reliance, communication, or interface component that conditions local reliance or off-aircraft transmission on readiness verification and applicable policy state. A governed connector may generate, verify, or enforce a transmission authorization decision and may control communication to crew interfaces, documentation interfaces, remote monitoring centers, or other downstream systems.

A “transmission authorization decision” is a machine-readable decision indicating whether a payload or output is allowed, denied, deferred, limited, quarantined, transformed, or permitted only in non-actionable form for transmission or other downstream reliance.

An “off-aircraft transmission payload” is telemetry, alerts, recommendations, summaries, readiness artifact information, governed context identifiers, policy identifiers, audit references, or other permitted information transmitted or prepared for transmission outside the aircraft cabin.

The term “off-aircraft communication link” refers to a satellite, air-to-ground, Wi-Fi, cellular, or other communication path used to transmit information outside the aircraft cabin or to a remote monitoring center.

A “remote monitoring center” is a remote service, facility, medical support center, telemedicine service, airline medical desk, hospital, emergency response center, ground support center, or other authorized location or system configured to receive, verify, review, store, display, document, forward, or act upon aircraft-cabin medical telemetry or related outputs.

The phrase “receiver-side readiness verification” refers to readiness verification performed by or for a receiving system, including a remote monitoring center, before a received payload is accepted, stored, displayed, forwarded, documented, presented to a clinician, or acted upon.

A “remote clinician interface” is an interface through which a remote clinician or authorized remote user may view verified telemetry, summaries, advisory information, source-quality information, identity confidence, readiness state, policy state, transformation history, or related information.

A “receipt-bound governance record” is an audit or governance record that binds a transmission authorization decision, payload, readiness artifact, remote monitoring destination, delivery status, acknowledgment, receiver-side verification outcome, acceptance, rejection, quarantine, retransmission request, or failed-readiness event.

The term “audit record” or “audit artifact” refers to a stored record of telemetry capture, source identity, identity resolution, consent state, memory atom creation, policy snapshot, gate evaluation, admissibility result, context bundle assembly, execution, readiness verification, transmission, delivery, receiver-side verification, proxy handoff, failure state, or downstream reliance. Audit records may be append-only, versioned, tamper-evident, signed, hashed, chained, or otherwise protected.

A “failure audit record” is an audit record associated with a device authentication failure, proxy node failure, gateway unavailability, policy evaluation failure, identity conflict, consent conflict, stale telemetry, memory gate denial, execution denial, readiness verification failure, governed connector unavailability, remote monitoring center rejection, receiver-side quarantine, RF degradation, satellite connectivity loss, fail-closed operation, or fail-quiet operation.

The term “flight-control systems” refers to aircraft systems involved in navigation, propulsion control, flight-control surfaces, autopilot control, flight management, aircraft operational control, or other flight-control functions.

The term “primary avionics buses” refers to aircraft data buses or avionics communication paths used for flight operations, aircraft control, aircraft navigation, aircraft monitoring, flight management, or primary avionics communication.

A “non-interference boundary” is a physical, logical, network, hardware, software, certification, or operational separation that prevents the aircraft-cabin medical telemetry system from commanding, altering, or interfering with flight-control systems, primary avionics buses, aircraft navigation, propulsion, or flight-control functions.

A “mediated cabin connectivity interface” is an interface that allows the cabin medical overlay network to use permitted cabin communication resources while enforcing authentication, authorization, encryption, payload filtering, readiness verification, destination control, policy control, or audit logging.

A “policy-mediated egress path” is a controlled communication path that allows information to leave the cabin medical overlay network only when applicable authentication, authorization, policy, readiness, destination, payload, or audit conditions are satisfied.

The term “RF coexistence controller” refers to a component, process, or logic configured to manage radio-frequency behavior of the aircraft-cabin medical telemetry system in view of aircraft cabin communication constraints. The RF coexistence controller may adjust transmit power, duty cycle, scanning interval, channel selection, retry behavior, backoff behavior, proxy selection, buffering, or fail-quiet operation.

The phrase “fail-closed operation” refers to a system response in which patient-specific execution, transmission, display as actionable information, documentation as verified information, remote monitoring acceptance, or other downstream reliance is denied, blocked, limited, or quarantined when required governance, readiness, authentication, policy, identity, consent, connectivity, or integrity conditions are not satisfied.

The phrase “fail-quiet operation” refers to a system response in which nonessential scanning, retries, transmission, alerting, or repeated output is reduced, suspended, suppressed, delayed, or limited to status information when continued operation could create interference, confusion, alarm fatigue, repeated failed messages, or unreliable output.

The phrase “fallback to standard cabin medical procedure” refers to a mode in which cabin crew or other authorized personnel are instructed or permitted to proceed using ordinary emergency medical kits, manual measurements, voice communication with ground support, onboard clinician assistance, operator-approved procedures, or other standard medical-event workflows when governed telemetry reliance is unavailable.

The phrase “diversion-related decision support” means advisory information, telemetry summaries, remote clinician input, readiness state, or related information that may support discussion or evaluation of whether aircraft diversion should be considered. Diversion-related decision support does not autonomously command an aircraft diversion or control aircraft operation.

### Architecture Overview and Aircraft Cabin Environment

In some embodiments, the disclosed system provides a governed medical telemetry architecture for use within an aircraft cabin. The system is configured to receive telemetry generated by wearable devices, portable medical devices, medical-grade devices, crew-carried devices, passenger-carried devices, dongles, adapters, or other cabin-available telemetry sources during a flight. The system processes the telemetry through a controlled sequence that includes capture, identity association, patient-partitioned storage, policy-based admission, governed context assembly, constrained execution, readiness verification, controlled reliance, and audit logging.

The aircraft cabin may be a passenger cabin of a commercial aircraft, private aircraft, charter aircraft, air ambulance, cargo aircraft configured for passenger transport, military aircraft, or other aircraft having a cabin area in which a passenger, crew member, patient, or other person may experience a medical event during flight. The cabin may include passenger seating, crew areas, galleys, lavatories, service carts, seat electronics, wireless access points, cabin communication equipment, emergency medical kits, passenger electronic devices, crew devices, and satellite or air-to-ground connectivity equipment.

The system may be deployed as a cabin medical overlay network. In some embodiments, the overlay network is installed as a retrofit system in an existing aircraft cabin. In other embodiments, the overlay network is provided as a portable emergency-use kit, a crew-device-supported system, a seat-associated system, a cabin gateway system, or a software-defined medical telemetry layer operating over permitted cabin communication resources. The system need not require structural modification of the aircraft cabin and need not require redesign or replacement of existing cabin systems.

The system may include a retrofit clinical integration plane configured to acquire telemetry from cabin-available devices and route the telemetry to governance components. The clinical integration plane may include proxy-capable aircraft cabin nodes, cabin telemetry gateways, device adapters, portable dongles, crew-carried devices, passenger mobile devices, edge processors, buffers, and communication interfaces. The clinical integration plane provides a controlled path between telemetry sources and patient-scoped governance components.

The system may associate received telemetry with context information before the telemetry is used for patient-specific monitoring or reasoning. The context information may include passenger identity, temporary patient profile, seat assignment, cabin location, flight identifier, device identifier, proxy node identifier, consent state, source time, receipt time, route information, jurisdiction information, source trust state, communication state, and transformation history. This association allows telemetry to be handled as governed patient-scoped information rather than as unbound device data.

The system may store telemetry and derived values as memory atoms in a patient-partitioned contextual memory. Patient partitioning allows the system to maintain separate contextual memory regions for different passengers or temporary patient profiles. In some embodiments, a flight-session memory region is keyed by flight identifier and passenger identity reference, temporary patient profile identifier, seat identifier, device identifier, or another partitioning value.

A monitoring task request may cause candidate memory atoms to be selected from the patient-partitioned contextual memory. Before any monitoring or reasoning component receives patient-scoped contextual input, a non-bypassable memory gate evaluates the candidate memory atoms under an aviation policy snapshot and integrity criteria. The aviation policy snapshot may represent operator rules, route-dependent rules, jurisdiction-dependent rules, consent rules, credentialing rules, retention rules, crew role permissions, remote clinician permissions, connectivity rules, emergency-mode rules, and flight-phase rules applicable to the requested task.

The non-bypassable memory gate may admit, deny, redact, abstract, attenuate, quarantine, or transform candidate memory atoms before execution. Admitted records and permitted transformed records are assembled into a governed context bundle. The governed context bundle may be bound to a policy snapshot identifier, bundle identifier, bundle digest, task identifier, passenger identity reference, and record of transformations. A monitoring or reasoning component is permitted to execute only through a constrained execution interface using the governed context bundle and an execution governance artifact.

Outputs from the monitoring or reasoning component do not automatically create operational reliance. In some embodiments, a readiness artifact is generated or verified before a downstream reliance event is permitted. The readiness artifact may be bound to the governed context bundle and the aviation policy snapshot. Downstream reliance events may include crew notification, display, documentation, storage as actionable medical information, physician coordination, transmission to a remote monitoring center, remote monitoring center acceptance, forwarding, or diversion-related decision support.

The system may include a governed connector that controls local reliance and off-aircraft transmission. The governed connector may condition satellite, air-to-ground, Wi-Fi, cellular, or other transmission of telemetry, alerts, recommendations, or summaries on successful readiness verification. A remote monitoring center may independently verify the readiness artifact before accepting, storing, displaying, forwarding, documenting, or acting upon a received payload.

The system is configured to preserve aircraft safety and crew authority. In some embodiments, the cabin medical overlay network is logically or physically isolated from flight-control systems and primary avionics buses. The system is not configured to command aircraft navigation, propulsion, flight-control, or primary avionics functions. The system may provide advisory information, readiness state, telemetry summaries, or remote monitoring information to crew or authorized personnel while standard cabin medical procedures and crew decision authority are preserved.

The system may adapt to aircraft cabin communication constraints. The system may adjust transmit power, duty cycle, scanning interval, channel selection, retry behavior, buffering behavior, proxy selection, or fail-quiet operation in response to cabin radio conditions, degraded wireless performance, gateway unavailability, satellite connectivity loss, or detected interference. If required governance, readiness, authentication, policy, or connectivity conditions are not satisfied, the system may fail closed, fail quiet, quarantine information, delay transmission, or fall back to standard cabin medical procedures.

### Retrofit Clinical Integration Plane and Proxy-Capable Cabin Nodes

In some embodiments, the system includes a retrofit clinical integration plane configured to operate within or in association with an aircraft cabin. The retrofit clinical integration plane provides a controlled acquisition layer between cabin-available telemetry sources and downstream governance components. The clinical integration plane may be installed in an existing aircraft cabin, supplied as a portable emergency-use system, implemented through crew-carried equipment, implemented through passenger-carried equipment under policy control, or implemented through a combination of fixed and portable components.

The retrofit clinical integration plane may include one or more proxy-capable aircraft cabin nodes. A proxy-capable aircraft cabin node may be any cabin-available component configured to receive, route, buffer, authenticate, normalize, or forward telemetry from a wearable device or medical device. The node may be fixed, portable, temporarily active, battery-powered, wall-powered, seat-powered, crew-operated, passenger-operated, dongle-based, adapter-based, gateway-based, or software-implemented.

In some embodiments, a proxy-capable aircraft cabin node is associated with a passenger seat or seat group. A seat-integrated node may be located at, under, adjacent to, or otherwise associated with a seat, seat row, service panel, seat power interface, seat electronics module, or cabin zone. The seat-integrated node may communicate with a wearable device, passenger mobile device, or medical device located near the associated seat. Seat association may be used as one input for identity resolution, location attribution, proxy selection, or provenance tracking.

In some embodiments, a crew-carried device functions as a proxy-capable aircraft cabin node. The crew-carried device may be a tablet, smartphone, handheld terminal, crew communication device, medical kit device, or other device operated by cabin crew. The crew-carried device may receive telemetry from a wearable device or medical device during an in-flight medical event, may assist with passenger identity verification, may collect crew observations, and may forward telemetry or contextual information into the clinical integration plane.

In some embodiments, a passenger mobile device executing a proxy application functions as a proxy-capable aircraft cabin node. The passenger mobile device may receive telemetry from a wearable device or medical device associated with the passenger and may forward the telemetry only when applicable authorization, consent, device association, communication, and aviation policy conditions are satisfied. The passenger mobile device may be limited to proxy operation and need not perform patient-specific monitoring, clinical decision-making, or readiness determination.

In some embodiments, a portable medical dongle or medical device adapter functions as a proxy-capable node or as part of a proxy-capable node. The dongle or adapter may connect to a wearable device, medical device, passenger mobile device, crew device, seat interface, emergency medical kit, or cabin gateway. The dongle or adapter may provide signal conversion, device identification, wired or wireless communication, local buffering, encryption, authentication, or data-format translation.

In some embodiments, a cabin telemetry gateway aggregates telemetry from multiple proxy-capable aircraft cabin nodes. The gateway may be installed in the aircraft cabin, carried on a medical cart, included in an emergency medical kit, mounted in a crew area, or otherwise positioned within the cabin. The gateway may receive telemetry from seat-associated nodes, crew-carried nodes, passenger mobile proxies, dongles, adapters, or medical devices and may route the telemetry to the telemetry ingestion subsystem.

The retrofit clinical integration plane may communicate with telemetry sources using one or more wired or wireless communication paths. The communication paths may include Bluetooth, Bluetooth Low Energy, Wi-Fi, wired serial communication, USB, Ethernet, near-field communication, optical communication, ultra-wideband, a proprietary medical device interface, a seat power data interface, or another communication path suitable for aircraft-cabin use. Different telemetry sources may use different communication protocols, and the clinical integration plane may normalize or convert those protocols into common telemetry objects.

The retrofit clinical integration plane may receive telemetry from wearable devices, passenger-worn devices, smart watches, pulse oximeters, blood pressure cuffs, portable ECG devices, temperature sensors, glucose monitors, respiratory sensors, infusion devices, ventilator devices, monitors deployed during an escalation event, or other cabin-available medical or physiologic devices. The received information may include physiologic measurements, waveforms, derived values, device status, alarm state, connection state, battery state, communication state, signal quality, and timing information.

Each proxy-capable aircraft cabin node may provide or preserve identifying information associated with telemetry capture. The identifying information may include a proxy node identifier, device identifier, device type, cabin location, seat association, crew device identifier, passenger mobile device identifier, dongle identifier, adapter identifier, communication path identifier, source time, receipt time, and link-quality state. This identifying information may be included in normalized telemetry objects, memory atoms, provenance records, audit records, or governed context bundles.

The retrofit clinical integration plane may support authentication and authorization of devices, nodes, adapters, dongles, applications, or gateways. If a device or proxy path cannot be authenticated or authorized, telemetry from that device or path may be denied, quarantined, assigned lower confidence, stored for audit only, or excluded from a governed context bundle. Authentication and authorization may be performed locally, by a cabin gateway, by an edge processing environment, by a policy service, or by a remote service when connectivity is available.

In some embodiments, the retrofit clinical integration plane includes cybersecurity controls for device trust, node trust, software integrity, and configuration integrity. A proxy-capable aircraft cabin node, dongle, adapter, cabin gateway, crew-carried device, passenger proxy application, or medical device interface may be required to present a device certificate, signed enrollment record, cryptographic credential, hardware identity, software identity, or attestation value before telemetry from that component is admitted for governed processing.

In some embodiments, a proxy-capable aircraft cabin node or gateway performs secure boot, verifies signed firmware, verifies signed software modules, verifies signed configuration files, or checks a trusted execution state before participating in telemetry capture or routing. If firmware, software, configuration, or execution state cannot be verified, the node may be excluded from proxy selection, assigned a reduced source trust classification, restricted to audit-only operation, or prevented from supplying patient-scoped telemetry to a governed context bundle.

The system may support credential rotation, key rotation, certificate expiration, revocation lists, compromised-device lists, anti-clone checks, device enrollment state, and signed configuration updates. A proxy-capable aircraft cabin node, dongle, adapter, mobile proxy application, gateway, or telemetry source may be revoked, quarantined, re-enrolled, or limited when a certificate expires, a key is rotated, a duplicate identity is detected, a configuration mismatch is detected, or a device identifier conflicts with an expected enrollment state.

In some embodiments, cybersecurity state is stored as part of device metadata, proxy node metadata, provenance metadata, memory atom metadata, admissibility result records, audit records, or failure audit records. Cybersecurity state may include certificate status, attestation result, secure-boot status, firmware version, signed-configuration version, revocation status, enrollment state, anti-clone result, key identifier, or trust classification. The non-bypassable memory gate may evaluate such cybersecurity state before admitting, denying, redacting, attenuating, quarantining, or transforming telemetry associated with the device or proxy path.

The retrofit clinical integration plane may include buffering. Buffering may occur in a proxy-capable cabin node, crew-carried device, passenger mobile device, dongle, adapter, cabin gateway, or edge processing environment. Buffered telemetry may be stored when a communication link is degraded, a gateway is unavailable, off-aircraft connectivity is unavailable, authentication is pending, a policy state is being evaluated, or a proxy handoff is in progress. Buffered telemetry may be transmitted or admitted later only if freshness, ordering, consent, policy, and readiness conditions are satisfied.

The retrofit clinical integration plane may operate in parallel with existing cabin systems. It need not replace passenger entertainment systems, passenger Internet systems, crew communication systems, emergency medical kits, or aircraft operational systems. When the clinical integration plane uses existing cabin connectivity as a transport path, telemetry and related payloads may remain subject to authentication, encryption, policy-mediated egress, readiness verification, and audit logging.

In some embodiments, the retrofit clinical integration plane is configured so that failure of the clinical integration plane does not disable standard cabin medical procedures or other cabin systems. If a node, dongle, adapter, gateway, communication link, or edge processor fails, the system may buffer data, lower confidence, suppress propagation, fail closed, fail quiet, generate an audit record, or instruct crew to follow standard cabin medical procedures.

### Telemetry Capture, Normalization, Proxy Selection, and Handoff

The system may receive telemetry from one or more wearable devices, portable medical devices, medical-grade devices, crew-carried devices, passenger-carried devices, dongles, adapters, or other telemetry sources available within the aircraft cabin. The telemetry may be received before, during, or after an in-flight medical event. In some embodiments, the system passively receives telemetry from a passenger-worn device. In other embodiments, telemetry is received after cabin crew deploys a portable medical device or connects a medical device adapter during an escalation event.

The telemetry may include physiologic measurements, device measurements, waveforms, device status, alarm state, operating state, connection state, battery state, communication state, signal quality, source time, event time, sequence information, or other data generated by or associated with the telemetry source. Examples of physiologic measurements include heart rate, oxygen saturation, respiratory rate, blood pressure, temperature, glucose-related data, ECG-derived information, activity state, perfusion-related data, or other monitored values.

A proxy-capable aircraft cabin node may receive a raw telemetry frame from a telemetry source. The raw telemetry frame may include a device-specific payload, device identifier, measurement value, units, device timestamp, sequence number, connection status, signal-quality indicator, alarm status, or other device-originated information. The proxy-capable aircraft cabin node, cabin telemetry gateway, edge processor, or telemetry ingestion subsystem may preserve the raw telemetry frame, convert it, or generate a normalized telemetry object.

A normalized telemetry object may include standardized fields for device identity, device type, proxy node identity, passenger or seat association, cabin location, flight identifier, event time, receipt time, measurement value, units, data quality, communication state, source trust state, and provenance. The normalized telemetry object may also include the original payload or a reference to the original payload when retention of raw source data is permitted by policy.

Normalization may include unit conversion, field mapping, timestamp normalization, source-path recording, signal-quality assignment, device-status mapping, or transformation of device-specific terms into common system terms. For example, different devices may report oxygen saturation, pulse rate, battery level, alarm state, or connection state using different formats. The telemetry ingestion subsystem may map those values into common fields that can be evaluated by the memory gate and later assembled into a governed context bundle.

The system may store source time, event time, receipt time, processing time, and validity interval separately when needed. Storing multiple time references allows the system to determine whether telemetry is current, stale, out-of-order, delayed, replayed, superseded, or inconsistent with other records. A later-received telemetry frame need not be treated as controlling if it reflects an older event time or if a more reliable source has superseded it.

When multiple proxy-capable aircraft cabin nodes can communicate with the same wearable device or medical device, the system may select an active proxy node. The active proxy node may be selected by a proxy selection controller located in a cabin node, cabin gateway, crew device, edge processing environment, clinical integration plane, or distributed combination of components. The proxy selection controller may evaluate communication metrics, location metrics, device metrics, policy conditions, and workflow conditions before selecting or reselecting the active proxy node.

Proxy selection may be based on link quality, received signal strength, packet loss, retry count, latency, channel quality, proximity, cabin location, seat association, node availability, node power state, device compatibility, authentication state, crew role, emergency state, passenger movement, medical-device deployment, or policy state. In some embodiments, a seat-associated node is selected during routine capture, while a crew-carried device or portable dongle is selected during a medical escalation event.

The system may identify one or more candidate proxy nodes. A candidate proxy node may be any node capable of communicating with the telemetry source and satisfying at least a minimum authorization or communication condition. The system may rank candidate proxy nodes according to link quality, proximity, authentication state, battery state, seat association, cabin location, or other criteria. The highest-ranked node, or another policy-preferred node, may be selected as the active proxy node.

A proxy handoff may occur when the active proxy node changes. Handoff may be triggered by passenger movement, device movement, seat change, node unavailability, degraded link quality, low battery state, authentication failure, communication loss, crew intervention, deployment of a medical-grade device, or a change in aviation policy snapshot. Handoff may also occur when a crew-carried device becomes a better proxy during an in-flight medical event.

During proxy handoff, the system may preserve provenance continuity. A provenance continuity record may identify the prior active proxy node, newly selected proxy node, device identifier, passenger identity reference or temporary patient profile, seat or cabin location, handoff time, handoff reason, link-quality state, telemetry sequence information, and any missing, duplicated, delayed, or replayed telemetry intervals. The provenance continuity record may later be included in memory atoms, audit records, or admissibility evaluations.

The system may continue telemetry capture during a proxy handoff when possible. If continuous capture is not possible, the system may buffer telemetry, mark a data gap, assign a lower confidence state, request confirmation, or prevent use of the affected interval for downstream reliance unless later policy conditions are satisfied. A memory atom associated with telemetry received after a handoff may include the updated proxy node identifier while also referencing the prior proxy path for continuity.

Telemetry buffering may occur before normalization, after normalization, before memory atom creation, before off-aircraft transmission, or during policy evaluation. A buffer may be maintained by a proxy node, passenger mobile device, crew-carried device, dongle, adapter, cabin gateway, edge processor, or clinical integration plane. Buffered telemetry may be stored with event time, receipt time, sequence number, device identifier, proxy node identifier, passenger or seat association, and freshness state.

Buffered telemetry may be replayed or forwarded after reconnection, proxy handoff, authentication restoration, gateway availability, or policy clearance. Before buffered telemetry is admitted for execution or downstream reliance, the system may evaluate freshness, ordering, consent state, source trust, passenger identity confidence, and policy status. Buffered data that is stale or otherwise unsuitable for reliance may be stored for audit only, denied, quarantined, abstracted, or transformed into a non-data-bearing record.

In some embodiments, the system distinguishes between telemetry received from consumer wearable devices and telemetry received from medical-grade devices. The distinction may affect source trust classification, admissibility, transformation, display, documentation, remote monitoring handling, or reliance conditions. For example, a wearable-derived trend may be admitted for preliminary advisory use while a medical-grade measurement may be assigned higher trust if device authentication, time validity, and patient association are confirmed.

The system may also detect conflicts among telemetry sources. A conflict may occur when two devices report inconsistent values, when a wearable device appears associated with a different passenger or seat, when a medical device is moved between passengers, when source time is inconsistent with flight or event time, or when a device state conflicts with crew observation. Conflicting telemetry may be flagged for memory gate evaluation, assigned a discrepancy or lower confidence state, quarantined, or transformed before execution.

The telemetry capture and normalization process therefore produces governed intermediate records rather than uncontrolled raw data. Each normalized telemetry object may carry sufficient device, proxy, passenger, seat, timing, communication, and provenance information to support later memory atom creation, policy evaluation, governed context bundle assembly, readiness artifact generation, audit logging, and remote verification.

### Passenger Identity Resolution, Consent, and Temporary Patient Profiles

The system associates telemetry with a passenger identity or temporary patient profile before the telemetry is used for patient-specific monitoring, reasoning, transmission, documentation, or other downstream reliance. Identity association may occur at initial telemetry capture, during telemetry ingestion, during memory atom creation, during a monitoring task request, during remote monitoring preparation, or during post-event reconciliation.

Passenger identity resolution may use one or more identity inputs. The identity inputs may include passenger manifest data, seat assignment data, boarding records, travel records, device association records, passenger mobile credentials, crew verification, companion or family confirmation, remote clinician confirmation, emergency tokens, temporary patient profiles, biometric verification where permitted, or post-event reconciliation information. The system need not require all identity inputs to be present. Different combinations of inputs may be used depending on aircraft configuration, operator policy, connectivity state, passenger condition, consent state, and emergency status.

Manifest data may identify a passenger scheduled or confirmed for the flight. Seat assignment data may identify an assigned seat, current seat, seat row, cabin zone, or crew-observed passenger location. Device association records may identify a wearable device, passenger mobile device, portable medical device, dongle, adapter, or other telemetry source associated with a passenger. A passenger mobile credential may include a boarding pass credential, airline application credential, wallet credential, medical credential, health application credential, or other electronically verifiable passenger-associated credential.

Crew verification may be used when electronic identity sources are incomplete, unavailable, conflicting, or insufficient. A crew member may confirm passenger name, seat, companion identity, consent status, device association, temporary profile, or observed condition. Crew verification may be stored with the crew member identity or role, timestamp, cabin location, verification type, and confidence state. Companion or family confirmation may be used when a passenger cannot communicate or when a companion has relevant identity information. Remote clinician confirmation may be used when a ground medical support service participates in identity confirmation or reconciliation.

The system may assign a passenger identity confidence state. The confidence state may indicate confirmed identity, probable identity, temporary identity, anonymous emergency partition, conflicting identity, pending reconciliation, or unresolved identity. The confidence state may be evaluated by the non-bypassable memory gate before telemetry is admitted for monitoring or reasoning. The confidence state may also affect transmission, display, documentation, remote monitoring acceptance, retention, or later reconciliation.

Consent may be captured or represented separately from identity. A consent state may indicate consent granted, consent denied, consent limited, consent revoked, consent unknown, emergency-use condition, or post-event confirmation pending. Consent may be captured through a passenger mobile application, crew interface, seat interface, travel record, medical kit workflow, emergency workflow, remote clinician workflow, biometric verification process where permitted, or another operator-approved process. The consent state may identify the scope of permitted activity, including capture, local processing, remote transmission, display, documentation, retention, or sharing with a remote monitoring center.

Consent state may be stored as contextual information and may be included in memory atoms, identity records, aviation policy snapshot evaluation, governed context bundles, readiness artifacts, or audit records. If consent is revoked or narrowed, the system may apply the revised consent state to later telemetry capture, memory gate evaluation, transmission, display, documentation, retention, or downstream reliance. Prior records may be retained, minimized, transformed, quarantined, or deleted according to the applicable aviation policy snapshot and retention rules.

In an emergency, identity or consent information may be unavailable, incomplete, or uncertain. The system may create a temporary patient profile. The temporary patient profile may include a temporary profile identifier, flight identifier, seat or cabin location, device association, proxy node association, crew observations, event time, consent state, emergency state, and available identity clues. The temporary patient profile allows telemetry to be stored in a patient-partitioned manner without waiting for final identity confirmation.

The system may also create an anonymous emergency patient partition. The anonymous emergency patient partition may be used when passenger identity cannot be reliably resolved or when policy requires temporary minimization. Telemetry stored in the anonymous emergency patient partition may be associated with device, proxy, seat, cabin location, flight, timing, consent, and provenance information while omitting or limiting identifying passenger information until reconciliation is permitted.

An emergency token may be generated to associate telemetry, crew observations, medical device data, remote consultation records, and transmission records with the same event or temporary patient profile. The emergency token may be generated by a crew interface, cabin gateway, clinical integration plane, passenger device, medical kit device, remote monitoring center, or other authorized component. The emergency token may later be linked to a confirmed passenger identity through post-event reconciliation.

Post-event reconciliation may occur during the flight, after connectivity is restored, after landing, or during later medical or operational review. Reconciliation may compare manifest data, seat assignment data, crew entries, device association records, emergency tokens, temporary profiles, remote monitoring records, timestamps, and audit records. Reconciliation may confirm an identity, correct an identity, merge records, separate records, or maintain an unresolved state. Reconciliation does not erase the original provenance of earlier records; instead, the system may preserve the original identity state, confidence state, reconciliation result, time of reconciliation, and basis for the change.

The system may prevent patient-scoped telemetry from being used outside the appropriate passenger identity or temporary profile. If a passenger moves seats, changes devices, is assisted by crew, or is monitored through a different proxy node, the system may update seat, location, device, and proxy information while preserving the patient association. If identity confidence becomes lower because of conflicting information, the system may quarantine affected telemetry, lower confidence, request crew confirmation, or prevent downstream reliance until the conflict is resolved.

Passenger identity resolution, consent handling, and temporary patient profile creation therefore allow aircraft-cabin telemetry to be associated with a controlled patient scope. The system does not rely solely on a device address, seat number, verbal report, or isolated crew entry. Instead, identity and consent are maintained as governed contextual states that can be evaluated before execution, transmission, documentation, remote monitoring, or other downstream reliance.

### Patient-Partitioned Contextual Memory and Memory Atoms

The system stores telemetry, derived values, identity information, consent information, device information, proxy information, and related contextual records in a patient-partitioned contextual memory. The patient-partitioned contextual memory maintains separation between records associated with different passengers, temporary patient profiles, or anonymous emergency patient partitions. The memory is not merely a general data store. It is structured so that later retrieval, gate evaluation, execution, transmission, and audit operations can be performed against a defined patient scope.

In some embodiments, the patient-partitioned contextual memory includes one or more flight-session memory regions. A flight-session memory region may be keyed by flight identifier, aircraft identifier, flight segment, passenger identity reference, temporary patient profile identifier, emergency token, seat identifier, device identifier, or a combination of such identifiers. The flight-session memory region may persist for a flight, for an event, for a policy-defined retention period, or for another period established by the aviation policy snapshot.

Each passenger or temporary patient profile may have a separate memory partition. A memory partition may store telemetry from wearable devices, portable medical devices, medical-grade devices, crew observations, remote clinician notes, consent events, device association events, proxy handoff records, transmission records, readiness verification records, and related audit information. A separate partition may also be created for an anonymous emergency patient when identity is not yet resolved or when policy requires identity minimization.

The system may support multiple passenger partitions within the same aircraft cabin. For example, if two passengers are monitored during the same flight, the system may maintain separate contextual memory regions for each passenger. Retrieval of patient-scoped information for one passenger does not expose records from another passenger unless an applicable aviation policy snapshot permits a specific aggregated, anonymized, emergency triage, or cabin-level review use.

Records stored in the patient-partitioned contextual memory may be represented as memory atoms. A memory atom is a structured contextual record containing telemetry or derived contextual information together with metadata used for provenance, timing, policy, identity, consent, transformation, and audit. A memory atom may represent a single measurement, a waveform segment, a device status event, an alarm event, a derived value, a crew observation, a consent update, a proxy handoff event, a remote clinician note, a transmission receipt, or a readiness verification result.

A memory atom may include a telemetry value field. The telemetry value field may store a measured physiologic value, device-generated value, waveform-derived value, signal-quality value, alarm value, or communication status value. A memory atom may also include a derived value field. The derived value field may store a trend, summary, risk indicator, normalized value, calculated value, confidence score, source-quality score, or other value produced from one or more measurements or contextual records.

A memory atom may include provenance metadata. Provenance metadata may identify the source device, source system, proxy-capable cabin node, cabin gateway, crew device, passenger mobile device, dongle, adapter, communication path, telemetry ingestion component, transformation component, user, remote service, or other origin of the record. Provenance metadata may also identify whether the record was received directly, buffered, replayed, transformed, manually entered, remotely received, or generated by a software component.

A memory atom may include time-validity metadata. Time-validity metadata may include source time, device time, event time, receipt time, processing time, validity interval, expiration time, sequence number, freshness state, stale-data state, or supersession state. Separate time values may be retained when a device reports one time, a proxy node receives the data at another time, and the clinical integration plane processes the data at a later time. This allows the system to determine whether a record remains suitable for monitoring, execution, transmission, documentation, or reliance.

A memory atom may include identity metadata. Identity metadata may include passenger identity reference, temporary patient profile identifier, anonymous emergency partition identifier, emergency token, identity confidence state, seat assignment, cabin location, boarding or manifest reference, crew verification reference, device association reference, or post-event reconciliation reference. Identity metadata allows the system to associate the record with the correct patient partition and to preserve identity uncertainty when identity is not yet confirmed.

A memory atom may include consent metadata. Consent metadata may include consent granted, consent denied, consent limited, consent revoked, consent unknown, emergency-use condition, consent source, consent time, consent scope, or consent expiration. Consent metadata may be evaluated before a memory atom is admitted into a governed context bundle, transmitted off aircraft, displayed, documented, retained, or presented to a remote monitoring center.

A memory atom may include device metadata. Device metadata may identify device identifier, device type, manufacturer, model, firmware version, authentication state, authorization state, battery state, communication state, signal quality, operating state, alarm state, source trust classification, calibration state, or device-quality state. Device metadata may be used to evaluate reliability, admissibility, and transformation requirements before the record is used.

A memory atom may include proxy node metadata. Proxy node metadata may identify the active proxy node, prior proxy node, candidate proxy nodes, proxy node identifier, proxy node type, proxy location, handoff event, link-quality metric, proximity metric, battery state, authentication state, and communication path. Proxy node metadata may preserve provenance continuity when telemetry is routed through different cabin nodes during a flight.

A memory atom may include seat or cabin-location metadata. Seat or cabin-location metadata may identify assigned seat, observed seat, current seat, seat row, cabin zone, aisle location, galley location, lavatory proximity, crew area, medical cart position, or other location context. This metadata may be used to support passenger identity resolution, proxy selection, crew workflow, and conflict detection.

A memory atom may include policy and transformation metadata. Policy metadata may identify the aviation policy snapshot applicable when the record was created, admitted, transformed, transmitted, or relied upon. Transformation metadata may identify normalization, unit conversion, redaction, abstraction, attenuation, quarantine, filtering, denial, admission, confidence adjustment, or transformation into a non-data-bearing form. Transformation metadata allows later components to determine how a record was changed before inclusion in a governed context bundle.

The patient-partitioned contextual memory may include access controls. Access controls may restrict retrieval by passenger identity, temporary patient profile, flight identifier, task type, role, policy state, consent state, and readiness state. A monitoring or reasoning component does not access the patient-partitioned contextual memory directly for patient-specific execution. Instead, candidate memory atoms are retrieved and evaluated by the non-bypassable memory gate before any governed context bundle is provided to the constrained execution interface.

In some embodiments, memory atoms are append-only or versioned. A later record may supersede an earlier record without deleting the earlier record. For example, a passenger identity may be corrected, a consent state may be revoked, a proxy node may change, or a telemetry value may be replaced by a medical-grade measurement. The system may preserve the earlier state and record the later correction, supersession, reconciliation, or transformation so that the chain of events remains auditable.

The patient-partitioned contextual memory may store both admitted and non-admitted records. Non-admitted records may include denied records, quarantined records, stale records, conflict records, unauthenticated-device records, or audit-only records. Such records may be retained for audit, review, troubleshooting, reconciliation, or later policy evaluation, but they are not made available as patient-scoped execution input unless admitted or transformed by the non-bypassable memory gate.

The memory structure therefore supports controlled use of aircraft-cabin medical telemetry. By storing telemetry as memory atoms in patient-partitioned contextual memory, the system preserves the information needed to determine who the telemetry relates to, where it came from, when it was generated, how it was routed, what policy applied, what consent state existed, what transformations occurred, and whether the record can be used for execution or downstream reliance.

### Aviation Policy Snapshot and Retrieval of Candidate Memory Atoms

An aviation policy snapshot represents the policy state used by the system when determining whether aircraft-cabin medical telemetry, derived values, contextual records, monitoring outputs, or transmission payloads may be used for a requested purpose. The aviation policy snapshot may be created before a flight, loaded during boarding, updated during flight, obtained from a cabin gateway, obtained from an operator system, obtained from a remote policy service when connectivity is available, or generated locally from stored policy rules.

The aviation policy snapshot may encode airline or aircraft-operator rules, route-dependent rules, jurisdiction-dependent privacy rules, consent rules, emergency-mode rules, credentialing constraints, crew role permissions, remote clinician permissions, escalation thresholds, retention rules, device trust rules, transmission rules, documentation rules, and flight-phase-dependent rules. The policy snapshot may also encode whether particular records may be admitted, denied, redacted, abstracted, attenuated, quarantined, transformed into non-data-bearing form, transmitted, displayed, stored, or relied upon.

In some embodiments, the aviation policy snapshot is bound to a flight, flight segment, passenger identity, temporary patient profile, cabin zone, route segment, jurisdictional region, communication state, or monitoring task. More than one aviation policy snapshot may be used during a flight. For example, a first policy snapshot may apply before an emergency mode is activated, a second policy snapshot may apply during emergency monitoring, and a third policy snapshot may apply after consent is revoked or after the aircraft enters another jurisdiction.

The aviation policy snapshot may be updated when a triggering condition occurs. Triggering conditions may include a route change, jurisdictional boundary crossing, flight phase change, passenger consent update, crew role change, remote monitoring availability change, satellite connectivity loss, escalation event, emergency mode activation, device trust change, operator policy update, or retention-rule change. When the policy snapshot changes, later memory-gate decisions, governed context bundles, execution governance artifacts, readiness artifacts, and audit records may be bound to the updated policy snapshot.

In some embodiments, a change in the aviation policy snapshot may invalidate, expire, suspend, or require reevaluation of a previously generated governed context bundle, execution governance artifact, readiness artifact, transmission authorization decision, or downstream reliance state. Such a change may occur when route, jurisdiction, flight phase, passenger consent state, identity confidence state, remote monitoring availability, device trust state, communication state, emergency-mode state, crew role, operator policy, or retention rule changes after the governed context bundle or readiness artifact was generated.

When such a triggering condition occurs, the system may mark the affected governed context bundle, execution governance artifact, readiness artifact, or transmission authorization decision as stale, superseded, expired, policy-mismatched, or pending reevaluation. The system may prevent patient-specific execution, off-aircraft transmission, remote monitoring acceptance, documentation, display as actionable medical information, or other downstream reliance until candidate memory atoms are reevaluated under the updated aviation policy snapshot.

In some embodiments, reevaluation includes retrieving the relevant candidate memory atoms, applying the updated aviation policy snapshot and integrity criteria, generating new admissibility result records, assembling a new governed context bundle, generating or updating an execution governance artifact, and generating or verifying a new readiness artifact. The system may preserve the prior bundle, artifact, and authorization decision for audit while preventing continued reliance on the superseded state.

The system may assign a policy snapshot identifier or digest to the aviation policy snapshot. The identifier or digest may be stored with memory atoms, admissibility result records, governed context bundles, execution governance artifacts, readiness artifacts, transmission authorization decisions, receipt-bound governance records, and audit records. Binding system outputs to the policy snapshot identifier or digest allows later verification of which policy state controlled a decision.

A monitoring task request may initiate retrieval of candidate memory atoms. The monitoring task request may be generated by a crew interface, proxy-capable cabin node, cabin telemetry gateway, clinical integration plane, remote monitoring center, remote clinician interface, rule-based trigger, alerting module, AI-assisted case manager, medical kit device, or other authorized component. The monitoring task request may request telemetry review, signal interpretation, alert generation, advisory output, documentation preparation, remote monitoring package creation, or another patient-scoped operation.

The monitoring task request may include or be associated with a task identifier. The task identifier may indicate the requested operation, requesting component, requesting user or role, flight identifier, passenger identity reference, temporary patient profile identifier, device identifier, time window, escalation state, consent state, remote monitoring state, or requested downstream reliance type. The task identifier may be stored in audit records and may be included in a governed context bundle, execution governance artifact, readiness artifact, or transmission record.

A retrieval engine may use the monitoring task request to select candidate memory atoms from the patient-partitioned contextual memory. The retrieval engine may select candidate memory atoms based on passenger identity, temporary patient profile, flight-session memory region, seat identifier, device identifier, proxy node identifier, telemetry type, source trust classification, event time, receipt time, validity interval, escalation state, or other retrieval criteria. The retrieval engine may also select associated contextual records, such as consent records, identity confidence records, device authentication records, proxy handoff records, remote clinician notes, or prior readiness verification outcomes.

Selection of candidate memory atoms is not an authorization for execution or reliance. The retrieval engine may identify records that are potentially relevant to the monitoring task, but those records remain subject to evaluation by the non-bypassable memory gate. A candidate memory atom may be relevant to the task but still denied, redacted, abstracted, attenuated, quarantined, or transformed under the aviation policy snapshot and integrity criteria.

The retrieval engine may preserve retrieval context. The retrieval context may include the task identifier, passenger identity reference, temporary patient profile identifier, flight identifier, retrieval time, retrieval criteria, selected memory atom identifiers, excluded memory atom identifiers, time windows, device filters, source trust filters, and the policy snapshot identifier or digest expected to govern the subsequent gate evaluation. This retrieval context may be stored in an audit record or passed to the non-bypassable memory gate.

In some embodiments, the retrieval engine may retrieve records from more than one patient partition only when permitted by the aviation policy snapshot. For example, an emergency triage task may request anonymized cabin-level status information, or a crew-facing dashboard may require a limited view of multiple active events. In such cases, the retrieval engine may select only policy-permitted records or transformed representations, and each passenger partition may still be separately evaluated by the non-bypassable memory gate.

The retrieval engine may also identify missing or incomplete information required for a requested task. For example, a task may require a recent oxygen saturation measurement, confirmed passenger identity, device authentication state, consent record, or medical-grade measurement. If the required information is not present, stale, conflicting, or unauthenticated, the retrieval engine may mark the missing condition for gate evaluation, request additional information, create an audit record, or allow the non-bypassable memory gate to deny, quarantine, or limit execution.

The aviation policy snapshot and retrieval process therefore define the policy and record-selection context for later execution. The system does not allow a monitoring or reasoning component to search freely through patient-partitioned contextual memory. Instead, the retrieval engine selects candidate memory atoms for a specific monitoring task, and the aviation policy snapshot supplies the policy state under which those candidate records must be evaluated before any patient-specific execution occurs.

### Non-Bypassable Memory Gate and Governed Context Bundle Assembly

A non-bypassable memory gate evaluates candidate memory atoms before patient-scoped contextual information is made available to a monitoring or reasoning component. The memory gate is positioned between the patient-partitioned contextual memory and any component that performs patient-specific monitoring, reasoning, alerting, summarization, documentation preparation, transmission preparation, or decision-support processing. The gate prevents a monitoring or reasoning component from obtaining patient-scoped context unless the relevant records have been evaluated under the applicable aviation policy snapshot and integrity criteria.

The non-bypassable memory gate may be implemented as a policy enforcement point, validation controller, admission controller, access-control service, context firewall, execution precondition service, policy filter, trust broker, gateway service, edge service, or another control function. The control function may be located in a cabin node, cabin telemetry gateway, clinical integration plane, edge processor, cloud service, remote monitoring service, policy server, trusted execution environment, or distributed combination of components. The particular software label or physical location is not limiting, provided that patient-scoped contextual input cannot be accessed by the monitoring or reasoning component without passing through the gate function.

The memory gate evaluates candidate memory atoms selected for a monitoring task. The evaluation may use an aviation policy snapshot, task identifier, passenger identity confidence state, consent state, device authentication state, source trust classification, telemetry type, time-validity information, proxy handoff information, transformation history, route or jurisdiction information, crew role, remote clinician authorization, communication state, and requested downstream reliance type. The evaluation may occur for each memory atom, for groups of memory atoms, for a candidate set as a whole, or for a combination of individual and group-level checks.

Integrity criteria applied by the memory gate may include provenance verification, temporal validity evaluation, source trust classification, conflict detection, completeness evaluation, device-authentication checking, proxy-node authentication checking, seat-identity consistency checking, consent-state checking, route-policy checking, jurisdiction-policy checking, communication-state checking, and transformation-history evaluation. Additional criteria may be used depending on operator rules, aircraft configuration, remote monitoring requirements, emergency status, or medical device type.

Provenance verification may determine whether a memory atom has a known source path. The source path may identify the original device, proxy node, cabin gateway, telemetry ingestion subsystem, transformation process, user entry, remote service, or software component that generated or handled the record. Temporal validity evaluation may determine whether the record is current, stale, expired, replayed, out-of-order, superseded, or outside the permitted time window for the requested task.

Source trust classification may distinguish medical-grade device data, consumer wearable data, passenger-device data, crew-entered observations, remote clinician notes, derived values, buffered records, replayed records, unauthenticated records, and audit-only records. Conflict detection may identify inconsistency between devices, inconsistency between telemetry and passenger identity, inconsistency between telemetry and seat or cabin location, inconsistency between consent state and proposed use, or inconsistency between a wearable-derived value and a medical-grade value. Completeness evaluation may determine whether the record includes required device, time, passenger, consent, proxy, seat, policy, and quality fields.

The memory gate may assign an admissibility state to each evaluated memory atom or to a candidate memory atom set. The admissibility state may include admission, denial, redaction, abstraction, attenuation, quarantine, or transformation into a non-data-bearing form. Admission permits the record to be included in a governed context bundle. Denial prevents the record from being used as patient-scoped context for the requested task. Redaction removes or masks a portion of the record. Abstraction may replace a specific value with a category, range, trend, or less identifying representation. Attenuation may reduce detail, confidence, precision, or downstream-use scope. Quarantine retains the record but prevents use for execution or reliance unless later conditions are satisfied. Transformation into a non-data-bearing form may retain a placeholder, denial reason, policy result, or audit marker without exposing patient-scoped data.

The memory gate may also generate an admissibility result record. The admissibility result record may include a memory atom identifier, patient partition identifier, passenger identity reference, task identifier, aviation policy snapshot identifier or digest, integrity criteria applied, admissibility state, transformation applied, denial reason, quarantine reason, confidence state, evaluator identifier, timestamp, rule identifier, and any associated conflict or missing-information indicator. The admissibility result record may be stored in an audit subsystem and may be referenced by a governed context bundle, execution governance artifact, readiness artifact, or receipt-bound governance record.

Records that are denied or quarantined are not supplied to a monitoring or reasoning component as patient-scoped execution input. In some embodiments, a denied or quarantined record may remain stored in patient-partitioned contextual memory for audit, troubleshooting, reconciliation, or later policy evaluation. In some embodiments, a non-data-bearing transformed record may be included to show that a record existed but was not available for execution because an admissibility condition was not satisfied.

After the memory gate completes evaluation, a governed context bundle assembler assembles admitted memory atoms and permitted transformed records into a governed context bundle. The governed context bundle is the controlled patient-scoped context made available for the requested monitoring task. The bundle may include admitted telemetry records, derived values, device status records, consent records, identity confidence records, proxy handoff records, admissibility results, transformation records, task identifiers, passenger identity references, flight identifiers, and aviation policy snapshot identifiers.

The governed context bundle may be assembled deterministically. Deterministic assembly may include fixed field ordering, sorted memory atom identifiers, normalized measurement units, standardized timestamp formats, consistent treatment of null or missing values, deterministic encoding of transformation records, and use of a defined canonical serialization process. Deterministic assembly allows later verification that the same admitted context was used for execution, readiness verification, transmission, and audit review.

The governed context bundle may include a bundle identifier. The bundle identifier may be generated from a sequence number, task identifier, passenger identity reference, flight identifier, event identifier, digest, random value, or combination of such values. The governed context bundle may also include a bundle digest computed over a canonical serialization of the bundle. The bundle digest may be used to detect alteration, mismatch, or substitution of the governed context.

The governed context bundle may include the identifier or digest of the aviation policy snapshot used for admission. Binding the governed context bundle to the aviation policy snapshot allows downstream components to determine whether execution, readiness verification, transmission, display, documentation, or remote monitoring relied on the same policy state that governed admission. If the policy state changes after bundle assembly, the system may require reevaluation, regeneration of the bundle, limited use, or denial of downstream reliance depending on the applicable policy.

The governed context bundle may include transformation records. A transformation record may identify whether a memory atom was redacted, abstracted, attenuated, converted to a non-data-bearing form, normalized, unit-converted, confidence-adjusted, quarantined, or excluded. The transformation record may also identify the rule or policy condition that caused the transformation. This allows a monitoring or reasoning component, readiness verifier, remote monitoring center, or audit reviewer to determine the scope and condition of the admitted context.

In some embodiments, the governed context bundle is the sole patient-scoped contextual input available to the monitoring or reasoning component for the task. The monitoring or reasoning component does not access patient-partitioned contextual memory directly and does not request raw telemetry outside the bundle. If the governed context bundle cannot be created, or if required candidate memory atoms are denied, quarantined, missing, stale, unauthenticated, or inconsistent, the system may deny execution, create a limited bundle, request additional information, provide non-patient-specific fallback content, or generate an audit record.

The non-bypassable memory gate and governed context bundle assembly therefore convert retrieved aircraft-cabin telemetry into a controlled execution context. The system does not treat retrieved records as automatically usable merely because they are present in memory. Instead, each record is evaluated under policy and integrity controls, admitted or transformed as appropriate, and assembled into a verifiable governed context bundle before patient-specific execution can occur.

### Constrained Execution and Execution Governance Artifacts

A constrained execution interface controls invocation of a monitoring or reasoning component. The constrained execution interface is positioned after governed context bundle assembly and before patient-specific execution. The interface prevents a monitoring or reasoning component from using raw telemetry, unadmitted memory atoms, denied records, quarantined records, records from another patient partition, or other patient-scoped contextual information outside the governed context bundle.

In some embodiments, execution is permitted only when a governed execution package is available. The governed execution package may include the governed context bundle and an execution governance artifact. The governed context bundle provides the admitted patient-scoped context for the monitoring task. The execution governance artifact provides machine-readable evidence that the governed context bundle was assembled under the applicable aviation policy snapshot and that the bundle is authorized for the requested execution.

The execution governance artifact may include a bundle identifier, bundle digest, aviation policy snapshot identifier or digest, task identifier, passenger identity reference, flight identifier, admissibility result records, transformation records, execution authorization state, expiration time, evaluator identifier, component identifier, signature, or other verification data. The execution governance artifact may be generated by the non-bypassable memory gate, governed context bundle assembler, policy service, clinical integration plane, cabin gateway, trusted execution environment, or other authorized component.

Before executing the monitoring or reasoning component, the constrained execution interface may verify the governed execution package. Verification may include checking that the governed context bundle is present, that the execution governance artifact is present, that the bundle identifier and bundle digest match, that the aviation policy snapshot identifier is current or otherwise permitted for the requested task, that the task identifier corresponds to the requested operation, that the passenger identity reference matches the patient partition, that the artifact has not expired, and that any required signature or authorization value is valid.

If the governed execution package is absent, invalid, expired, mismatched, incomplete, unsigned when a signature is required, inconsistent with the aviation policy snapshot, or inconsistent with the requested monitoring task, the constrained execution interface may deny execution. In some embodiments, the interface may delay execution, quarantine the task, request additional identity confirmation, request updated consent, request updated device authentication, request updated policy evaluation, or route the task for review. The system may generate an execution denial record or other audit record identifying the reason execution did not proceed.

In some embodiments, the constrained execution interface may permit limited execution when full patient-specific execution is not allowed. Limited execution may include non-patient-specific fallback content, general emergency procedure prompts, crew checklist information, device troubleshooting guidance, or instructions to obtain additional measurements or verification. Limited execution may exclude patient-scoped telemetry, patient identity information, or other data that has not been admitted through the non-bypassable memory gate.

The monitoring or reasoning component may include a rule engine, threshold detector, signal-processing module, trend module, predictive model, AI-assisted case manager, alert generator, summarization module, documentation-preparation module, remote monitoring package generator, or another software or hardware component configured to process admitted patient-scoped context. The monitoring or reasoning component may be local to the aircraft cabin, located in a cabin gateway, located in a crew device, distributed across cabin and remote components, or operated by a remote monitoring service.

The monitoring or reasoning component receives the governed context bundle as its patient-scoped input. In some embodiments, the component receives no direct credentials or interface that would allow unrestricted access to the patient-partitioned contextual memory. In other embodiments, the component may call an access service, but the access service returns only records admitted into the governed context bundle or otherwise permitted by the same gate and policy controls. This prevents the component from bypassing admission controls through alternate memory queries or raw telemetry paths.

The monitoring or reasoning component may generate an output. The output may include an advisory output, alert, recommendation, telemetry summary, trend, risk indication, confidence indication, source-quality statement, documentation draft, remote monitoring summary, or transmission payload. The output may include references to the governed context bundle, execution governance artifact, policy snapshot, task identifier, passenger identity reference, or admitted memory atom identifiers used to produce the output.

The output of the monitoring or reasoning component is not automatically treated as a permitted downstream reliance event. Generation of an output does not by itself authorize crew notification, display, documentation, off-aircraft transmission, remote monitoring center acceptance, physician coordination, forwarding, or diversion-related decision support. Those downstream reliance events remain subject to readiness artifact verification and governed connector control.

In some embodiments, the constrained execution interface records an execution audit record. The execution audit record may identify the monitoring task request, governed context bundle, execution governance artifact, monitoring or reasoning component, component version, model identifier if applicable, execution time, execution result, output identifier, denial or fallback condition, and any exception encountered during execution. The execution audit record may be stored with other audit artifacts to support reconstruction of the path from telemetry capture to downstream reliance.

The constrained execution interface therefore changes the technical execution path of monitoring and reasoning components. Instead of allowing a component to retrieve telemetry directly from memory or device sources, the system requires execution to occur only after admitted records are assembled into a governed context bundle and bound to an execution governance artifact. This structure reduces the risk that stale, misidentified, unauthenticated, unconsented, conflicting, or policy-prohibited telemetry will be used as patient-specific input.

### Readiness-Conditioned Reliance, Governed Connector, and Remote Monitoring

Outputs generated by a monitoring or reasoning component are controlled before they are used in a downstream reliance event. A downstream reliance event may include crew notification, display, rendering, storage as actionable medical information, documentation, physician coordination, off-aircraft transmission, remote monitoring center acceptance, forwarding, or diversion-related decision support. The system does not treat a monitoring output, alert, recommendation, summary, or generated advisory as automatically reliable merely because the output was produced by an authorized component.

In some embodiments, a readiness artifact is generated or verified before a downstream reliance event is permitted. The readiness artifact may be a machine-readable control object bound to the governed context bundle and the aviation policy snapshot. The readiness artifact may include a bundle identifier, aviation policy snapshot identifier, time reference, verification digest, digital signature, nonce, evaluator identifier, execution certificate, readiness certificate, policy-verification result, transmission authorization record, or other verification data.

The readiness artifact may be generated by a cabin gateway, clinical integration plane, non-bypassable memory gate, governed context bundle assembler, constrained execution interface, policy service, readiness verification service, governed connector, trusted execution environment, or remote monitoring service. In some embodiments, different components may generate and verify different parts of the readiness artifact. For example, a local component may generate a bundle digest and policy identifier, while a remote service verifies the signature or policy state before accepting a transmitted payload.

Readiness verification may include confirming that the readiness artifact is present, unexpired, properly signed when a signature is required, bound to the governed context bundle used for execution, bound to the applicable aviation policy snapshot, consistent with the execution governance artifact, consistent with the task identifier, consistent with the passenger identity reference, and valid for the requested reliance type. Verification may also include checking consent state, remote monitoring availability, route or jurisdiction rules, crew role, device trust state, and whether the output is permitted for display, documentation, transmission, or clinical review.

If readiness verification fails, the downstream reliance event may be denied, deferred, quarantined, limited, or prevented. For example, the system may prevent a payload from being transmitted to a remote monitoring center, prevent a generated alert from being displayed as actionable medical information, prevent documentation as a verified clinical record, prevent forwarding to another system, or limit the output to non-patient-specific fallback guidance. The system may also generate a failed-readiness event record and route the issue for crew review, policy review, additional identity confirmation, device authentication, updated consent, or retransmission.

A governed connector may control local reliance and off-aircraft transmission. The governed connector may be implemented as a software service, gateway function, policy-mediated egress component, communication controller, transmission service, remote monitoring interface, or combination of such components. The governed connector receives the monitoring or reasoning output and the readiness artifact, verifies or requests verification of the readiness artifact, and determines whether a downstream reliance event may proceed.

In some embodiments, when the system operates in a governed mode, the governed connector provides the sole permitted egress path for patient-scoped telemetry, monitoring outputs, reasoning outputs, alerts, recommendations, summaries, documentation-ready records, readiness artifacts, execution governance references, governed context identifiers, or related medical-event payloads directed to approved downstream destinations. Patient-scoped telemetry or generated outputs are not transmitted, forwarded, displayed as actionable medical information, documented as verified medical information, or made available to a remote monitoring center through an alternate uncontrolled path.

The governed connector may block, deny, quarantine, or redirect an attempted transmission or downstream reliance event that does not include a valid readiness artifact, transmission authorization decision, permitted destination, permitted payload type, policy snapshot identifier, and audit reference. In some embodiments, the governed connector intercepts or mediates attempted egress through satellite, air-to-ground, Wi-Fi, cellular, cabin network, crew-device, passenger-device, documentation-system, or remote-monitoring interfaces and permits only those payloads that satisfy the applicable aviation policy snapshot and readiness verification conditions.

If an alternate communication path is detected or requested for patient-scoped telemetry or generated medical-event output, the system may require re-routing through the governed connector, generate a failure audit record, notify an authorized crew interface, quarantine the payload, or limit the output to non-patient-specific fallback content. This prevents off-aircraft transmission or downstream reliance from bypassing the governed context bundle, execution governance artifact, readiness artifact, and audit controls.

The governed connector may generate a transmission authorization decision. The decision may allow, deny, defer, limit, quarantine, or permit only a non-actionable version of a payload. The decision may depend on readiness artifact verification, aviation policy snapshot state, passenger consent state, passenger identity confidence, device trust state, communication state, payload type, intended destination, route or jurisdiction rules, remote monitoring availability, and emergency-mode status.

When off-aircraft transmission is permitted, the governed connector may transmit telemetry, alerts, recommendations, summaries, readiness artifacts, execution governance references, bundle identifiers, policy identifiers, or other permitted payload information to a remote monitoring center. Transmission may occur through satellite, air-to-ground, Wi-Fi, cellular, or another available communication path. The transmitted payload may be minimized, encrypted, signed, redacted, abstracted, compressed, or otherwise transformed according to policy.

The governed connector may also determine that transmission should be delayed. Delay may occur when off-aircraft connectivity is unavailable, satellite bandwidth is limited, the remote monitoring center is unavailable, consent is pending, identity confidence is insufficient, readiness verification is incomplete, or the aviation policy snapshot does not permit immediate egress. In such cases, the system may buffer the payload, store a pending-transmission state, provide crew status, and transmit later if freshness, ordering, consent, policy, and readiness conditions remain satisfied.

A remote monitoring center may receive a transmitted payload. The remote monitoring center may be operated by an airline, medical support service, hospital, telemedicine provider, emergency response center, aircraft operator, or other authorized entity. In some embodiments, the remote monitoring center independently verifies the readiness artifact before accepting, storing, displaying, forwarding, documenting, or acting upon the payload.

Receiver-side readiness verification may include checking the bundle identifier, aviation policy snapshot identifier, time reference, digest, digital signature, nonce, evaluator identifier, execution governance artifact reference, transmission authorization record, and payload identity. Receiver-side verification may also check whether the payload was received from an authorized governed connector, whether the readiness artifact corresponds to the transmitted payload, whether the payload is current, and whether the requested remote monitoring function is permitted by policy.

If receiver-side verification succeeds, the remote monitoring center may accept the payload for permitted use. The accepted payload may be stored, displayed to a remote clinician, associated with a remote monitoring event, documented, or used for physician coordination according to policy. A remote clinician interface may present telemetry, summaries, source quality, identity confidence, readiness state, policy state, timing information, or transformation history to a remote clinician.

If receiver-side verification fails, the remote monitoring center may reject the payload, place the payload in a quarantined store, request retransmission, request a corrected readiness artifact, request updated policy information, or record a failed-readiness event. In some embodiments, a quarantined payload is not presented as actionable medical information until the readiness issue is resolved. In other embodiments, the remote monitoring center may retain the failed payload only for audit, troubleshooting, or reconciliation.

The system may also control local reliance in the aircraft cabin. A crew interface, cabin display, crew display, or documentation interface may receive only the information permitted by readiness verification and the aviation policy snapshot. The system may display a readiness state, transmission state, source-quality state, identity confidence state, or failure state to cabin crew. If patient-specific reliance is not permitted, the crew interface may display non-patient-specific fallback guidance or instructions to follow standard cabin medical procedures.

In some embodiments, the system supports physician coordination and diversion-related decision support. The system may transmit or present medical telemetry summaries, advisory outputs, remote clinician comments, readiness state, and audit references to authorized users. The system does not autonomously divert an aircraft and does not command flight-control or aircraft operational systems. Any diversion-related support remains advisory and subject to crew authority, operator procedures, and applicable aviation rules.

Readiness-conditioned reliance therefore provides a second control point after constrained execution. The first control point governs what patient-scoped context may be used for monitoring or reasoning. The second control point governs whether the resulting output may be relied upon, transmitted, displayed, documented, accepted by a remote monitoring center, or used in a downstream medical or operational workflow.

### Audit, Traceability, and Receipt-Bound Governance Records

The system may generate and store audit records that preserve the path from telemetry capture to downstream reliance. Audit records may be generated at multiple stages, including telemetry receipt, proxy selection, proxy handoff, telemetry normalization, identity resolution, consent capture, memory atom creation, aviation policy snapshot selection, memory gate evaluation, governed context bundle assembly, constrained execution, readiness artifact generation, governed connector transmission, remote monitoring center verification, and downstream reliance.

An audit record may identify the source of telemetry or contextual information. Source information may include a device identifier, device type, proxy node identifier, cabin gateway identifier, dongle identifier, adapter identifier, crew device identifier, passenger mobile device identifier, user identity, crew role, remote service identifier, software component identifier, or communication path identifier. Source information may also indicate whether the record was received directly, buffered, replayed, manually entered, software-generated, remotely received, or transformed.

An audit record may include identity and patient-partition information. Identity-related audit information may include passenger identity reference, temporary patient profile identifier, anonymous emergency patient partition identifier, emergency token, seat identifier, cabin location, identity confidence state, identity source, crew verification record, companion confirmation record, remote clinician confirmation record, or post-event reconciliation result. These records allow later review of how telemetry was associated with a passenger or temporary patient profile.

An audit record may include consent and policy information. Consent-related audit information may include consent state, consent source, consent time, consent scope, consent limitation, consent revocation, emergency-use condition, or consent reconciliation. Policy-related audit information may include aviation policy snapshot identifier or digest, policy source, policy version, route or jurisdiction state, crew role rule, remote monitoring rule, retention rule, emergency-mode rule, and any policy update that affected admission, transmission, display, documentation, or reliance.

An audit record may include timing information. Timing information may include source time, device time, event time, proxy receipt time, gateway receipt time, processing time, retrieval time, gate evaluation time, execution time, readiness verification time, transmission time, delivery time, receipt time, remote verification time, quarantine time, and reconciliation time. Maintaining separate time references allows the system to reconstruct whether information was current, stale, delayed, replayed, superseded, or timely when used.

The system may store admissibility result records. An admissibility result record may identify a memory atom or candidate memory atom set, the task identifier, the aviation policy snapshot used, the integrity criteria applied, the admissibility state assigned, any transformation applied, any denial or quarantine reason, any conflict detected, any missing information condition, and the evaluator or component that performed the evaluation. The admissibility result record may be referenced by a governed context bundle, execution governance artifact, readiness artifact, or later audit review.

The system may store governed context bundle records. A governed context bundle record may include a bundle identifier, bundle digest, task identifier, passenger identity reference, flight identifier, policy snapshot identifier or digest, included memory atom identifiers, excluded memory atom identifiers, transformation records, admissibility result references, and canonical serialization information. These records allow the system or a reviewer to determine what patient-scoped context was available to a monitoring or reasoning component.

The system may store execution records. An execution record may include the governed execution package identifier, governed context bundle identifier, execution governance artifact identifier, monitoring or reasoning component identifier, component version, model identifier where applicable, task identifier, execution time, execution result, output identifier, denial condition, fallback condition, exception condition, or quarantine condition. Execution records may show whether a component executed on admitted context or whether execution was denied or limited.

The system may store readiness and reliance records. A readiness record may include a readiness artifact identifier, bundle identifier, policy snapshot identifier, time reference, digest, signature, nonce, evaluator identifier, verification result, expiration state, permitted reliance type, and failure reason if verification failed. A reliance record may identify whether crew notification, display, documentation, storage as actionable medical information, off-aircraft transmission, remote monitoring center acceptance, forwarding, physician coordination, or diversion-related support was allowed, denied, deferred, limited, quarantined, or prevented.

The system may store transmission records. A transmission record may identify the governed connector, payload identifier, payload type, destination, communication path, transmission authorization decision, readiness artifact, policy snapshot identifier, encryption state, redaction state, transmission time, delivery status, retry status, delayed transmission state, or failure state. Transmission records may be used to determine whether a payload left the aircraft cabin, when it was sent, what readiness state applied, and whether transmission was permitted.

The system may store receiver-side verification records. A receiver-side verification record may be generated by or received from a remote monitoring center. The record may identify the transmitted payload, readiness artifact, bundle identifier, policy snapshot identifier, signature or digest check, remote monitoring center identifier, remote verifier identifier, acceptance state, rejection state, quarantine state, retransmission request, failed-readiness event, or remote clinician presentation state.

A receipt-bound governance record may be stored in response to a delivery receipt, acknowledgment, delivery status indication, readiness verification outcome, receiver-side quarantine event, failed-readiness event, or remote monitoring center verification result. The receipt-bound governance record may bind the original transmission authorization decision to the later delivery or verification result. In some embodiments, the receipt-bound governance record includes the payload identifier, readiness artifact identifier, remote monitoring center identifier, delivery time, verification time, acceptance or rejection status, quarantine status, retransmission request, and audit references.

The system may store proxy handoff and communication records. A proxy handoff record may identify the prior proxy node, newly selected proxy node, device identifier, passenger identity reference, handoff time, handoff reason, link-quality state, battery state, authentication state, cabin location, and telemetry sequence continuity. Communication records may identify packet loss, retries, degraded wireless conditions, interference events, buffering, delayed forwarding, satellite connectivity loss, and fail-quiet operation.

The system may store failure records. Failure records may include device authentication failure, proxy node failure, gateway unavailability, policy evaluation failure, identity conflict, consent conflict, stale telemetry, memory gate denial, execution denial, readiness verification failure, governed connector unavailability, remote monitoring center rejection, receiver-side quarantine, or inability to complete off-aircraft transmission. Failure records may identify the system response, such as denial, quarantine, delayed transmission, fallback content, fail-closed operation, fail-quiet operation, or crew status notification.

Audit records may be append-only, versioned, tamper-evident, signed, hashed, or otherwise protected from unauthorized alteration. In some embodiments, the system computes digests over selected records, stores chained audit entries, signs readiness or transmission records, or records policy identifiers so that later review can verify the integrity of the audit trail.

The audit subsystem may support reconstruction of the full event path. A reviewer may determine which telemetry was captured, which passenger partition was used, which identity and consent states applied, which policy snapshot governed the event, which memory atoms were admitted or denied, which governed context bundle was used for execution, which readiness artifact authorized reliance, whether a payload was transmitted, whether a remote monitoring center accepted or rejected the payload, and what information was presented to crew or a remote clinician.

The audit, traceability, and receipt-bound governance records therefore provide evidence of system behavior. They show not only what medical data was captured, but also how the data was associated, governed, admitted, transformed, executed upon, transmitted, verified, accepted, rejected, quarantined, or relied upon.

### Cabin Isolation, RF Coexistence, Fail-Closed Operation, and Multiple Passenger Handling

In some embodiments, the aircraft-cabin medical telemetry system operates as a cabin medical overlay network that is isolated from flight-control systems and primary avionics buses. The system may use cabin-available communication resources, crew devices, passenger devices, proxy-capable cabin nodes, dongles, gateways, or portable medical equipment, but it is not configured to command, alter, or interfere with aircraft navigation, propulsion, flight-control, or primary avionics functions.

Isolation may be implemented by physical separation, network segmentation, firewall rules, policy-mediated interfaces, one-way communication paths, hardware isolation, software access controls, absence of avionics interfaces, operator configuration, or operational procedures. In some embodiments, the system may communicate with cabin connectivity infrastructure only through a mediated cabin connectivity interface. The mediated interface may enforce authentication, authorization, encryption, payload filtering, readiness verification, destination control, and audit logging.

The system may coexist with in-flight entertainment networks, passenger Internet access networks, crew communication systems, satellite connectivity systems, seat electronics, passenger devices, and other cabin systems. Coexistence does not require the cabin medical overlay network to become part of those systems. Where a cabin communication resource is used as a transport path, the medical telemetry payload may remain governed, encrypted, policy-controlled, and subject to readiness verification before egress.

The system may include a policy-mediated egress path for off-aircraft transmission or downstream communication. The egress path may allow telemetry, alerts, recommendations, summaries, readiness artifacts, receipt records, or audit records to leave the cabin medical overlay network only when applicable governance and readiness conditions are satisfied. In some embodiments, the egress path is configured to prevent uncontrolled inbound commands and to prevent ungoverned patient-scoped data from being transmitted to non-approved destinations.

The system may adapt to the aircraft cabin radio environment. The cabin radio environment may include wireless access points, passenger electronic devices, crew devices, wearable devices, medical devices, seat electronics, service carts, and other RF sources. An RF coexistence controller may monitor or infer radio conditions and adjust operation to reduce interference, preserve telemetry quality, and avoid unnecessary transmissions.

RF coexistence control may include transmit power control, duty-cycle control, scanning interval control, adaptive channel selection, retry and backoff control, proxy selection, buffering, or temporary suspension of nonessential communication. The system may reduce transmit power, reduce scan frequency, change channel, increase backoff, select a different proxy node, buffer telemetry locally, delay noncritical transmission, or enter fail-quiet operation when radio conditions degrade.

The system may detect degraded wireless performance. Degraded wireless performance may include low signal strength, high packet loss, excessive retries, unstable proxy connections, incomplete telemetry frames, channel congestion, increased latency, communication collisions, or interference with other cabin communication activity. The system may mark affected telemetry with lower confidence, create a failure audit record, request a different proxy node, delay transmission, or prevent downstream reliance on affected records unless later policy conditions are satisfied.

The system may detect off-aircraft connectivity loss or degradation. Satellite, air-to-ground, Wi-Fi, cellular, or other connectivity may be unavailable, delayed, bandwidth-limited, restricted by route or flight phase, or unavailable because of equipment state. When connectivity is unavailable, the system may buffer telemetry, summaries, readiness artifacts, transmission payloads, and audit records. Buffered records may be transmitted later only if freshness, ordering, consent, policy, and readiness conditions remain satisfied.

The system may fail closed when required governance, readiness, authentication, policy, identity, consent, or connectivity conditions are not satisfied. Fail-closed operation may include denying patient-specific execution, denying off-aircraft transmission, preventing display as actionable medical information, preventing documentation as verified medical information, blocking remote monitoring center acceptance, quarantining a payload, or storing a record for audit only. Fail-closed operation may preserve the underlying record while preventing unauthorized or unreliable reliance.

The system may fail quiet when continued scanning, retries, alerting, or transmission would create interference, confusion, excessive crew burden, repeated failed messages, or unreliable output. Fail-quiet operation may reduce nonessential radio activity, suppress repeated failed transmissions, suspend noncritical alerts, limit outputs to status messages, or require manual crew action before further patient-specific processing. Fail-quiet operation may be used in response to RF interference, repeated readiness failures, unavailable governed connector, degraded communication, or device authentication failure.

If the governed telemetry path is unavailable, the system may fall back to standard cabin medical procedures. Fallback may include instructing crew to use onboard emergency medical kits, obtain manual measurements, request assistance from onboard medical personnel, contact ground-based medical support by voice when available, follow operator emergency procedures, or continue other approved workflows. The fallback does not prevent standard medical assistance and does not transfer aircraft command or medical authority to the telemetry system.

The system may generate failure audit records when fail-closed, fail-quiet, degraded communication, authentication failure, readiness failure, or governed connector unavailability occurs. A failure audit record may include time, flight identifier, passenger identity reference or temporary profile identifier, device identifier, proxy node identifier, communication path, policy snapshot identifier, readiness artifact identifier, attempted action, failure condition, system response, and crew status indication.

The system may provide crew status indications. Crew status indications may show that telemetry capture is active, a device is unauthenticated, identity confidence is low, readiness verification failed, transmission is delayed, payload is quarantined, connectivity is unavailable, fail-quiet mode is active, or standard procedures should be followed. Crew status indications may be role-limited and may display only information permitted by the aviation policy snapshot.

The system may support simultaneous monitoring of multiple passengers. Separate patient partitions may be maintained for separate passengers, temporary patient profiles, or anonymous emergency patient partitions. Telemetry, identity records, consent records, proxy handoff records, memory atoms, governed context bundles, readiness artifacts, transmission records, and audit records may be maintained separately for each patient partition.

For multiple-passenger operation, the retrieval engine may select candidate memory atoms separately for each passenger partition. The non-bypassable memory gate may independently evaluate candidate records for each passenger under the applicable aviation policy snapshot. The governed context bundle assembler may generate distinct governed context bundles, and the readiness verifier may generate or verify distinct readiness artifacts for each passenger identity or temporary patient profile.

The system may prevent commingling of patient-scoped information between passenger partitions. A monitoring or reasoning component invoked for one passenger may receive only the governed context bundle for that passenger unless the aviation policy snapshot permits an aggregated, anonymized, emergency triage, or cabin-level review use. If such a use is permitted, the system may transform or minimize the underlying records before use.

Multiple passenger monitoring may also include independent audit and transmission handling. One passenger’s payload may be transmitted to a remote monitoring center while another passenger’s payload is held, quarantined, or denied because of different consent state, identity confidence, device authentication state, readiness artifact state, or policy condition. This allows simultaneous in-flight events to be handled without treating the aircraft cabin as a single undifferentiated patient context.

The isolation, RF coexistence, fail-closed, fail-quiet, and multiple passenger features provide aircraft-specific controls for the telemetry architecture. They allow the system to operate in a constrained cabin environment, use available cabin communication resources, avoid interference with aircraft operational systems, preserve crew authority, and maintain patient-specific separation even when multiple passengers, devices, proxy nodes, and communication paths are active.

### Example Operational Sequence and Technical Effects

In one example, a passenger boards an aircraft while wearing a wearable device capable of generating physiologic telemetry. The passenger is associated with a manifest record and a seat assignment. The wearable device is detected by a seat-associated proxy-capable aircraft cabin node or by a passenger mobile device executing a proxy application. The system associates the wearable device with the passenger using one or more of the seat assignment, device association, passenger mobile credential, consent state, or crew verification.

During flight, the wearable device generates telemetry. The telemetry may include heart rate, oxygen saturation, rhythm-related information, activity state, signal quality, device state, or other monitored information. The proxy-capable aircraft cabin node receives the telemetry, timestamps or forwards the telemetry, and associates the telemetry with a device identifier and proxy node identifier. The telemetry is provided to the retrofit clinical integration plane and converted into normalized telemetry objects.

The telemetry ingestion subsystem associates the normalized telemetry objects with passenger, flight, seat, device, proxy, consent, timing, and provenance information. The resulting records are stored as memory atoms in a patient-partitioned contextual memory associated with the passenger. Each memory atom may include a telemetry value or derived value together with metadata indicating the source device, active proxy node, event time, receipt time, consent state, location context, source trust classification, and transformation history.

If the passenger experiences a medical event, cabin crew may initiate an escalation workflow. The crew may deploy a portable medical device, such as a pulse oximeter, blood pressure cuff, portable ECG device, thermometer, or other medical device. The portable medical device may communicate through a crew-carried device, portable medical dongle, medical device adapter, seat-associated node, or cabin telemetry gateway. The system may update the passenger partition with new memory atoms corresponding to the medical-device telemetry and crew observations.

If the passenger moves from the assigned seat, if crew carries a medical device to another cabin location, or if link quality changes, the system may perform proxy handoff. A proxy selection controller may select a different proxy-capable aircraft cabin node based on link quality, proximity, authentication state, battery state, cabin location, device compatibility, or policy state. The system stores a provenance continuity record identifying the prior proxy node, newly selected proxy node, handoff time, handoff reason, device identifier, passenger reference, and telemetry continuity state.

A monitoring task request may be generated by a crew interface, cabin gateway, rule-based trigger, remote monitoring workflow, or AI-assisted case manager. The retrieval engine selects candidate memory atoms from the passenger’s partition. The selected candidate memory atoms may include wearable telemetry, medical-grade device telemetry, device status, proxy handoff records, identity confidence records, consent records, and crew observations.

The non-bypassable memory gate evaluates the candidate memory atoms under the applicable aviation policy snapshot. The policy snapshot may reflect the flight route, jurisdiction, operator rules, consent state, crew role, escalation status, remote monitoring availability, and connectivity state. The memory gate applies integrity criteria, including provenance verification, temporal validity, source trust classification, conflict detection, completeness, device authentication, seat-identity consistency, consent status, and route-policy status.

The memory gate admits records that satisfy the policy and integrity criteria. It may deny, redact, abstract, attenuate, quarantine, or transform other records. For example, wearable telemetry may be admitted as a trend while a stale value may be denied, an unauthenticated device record may be quarantined, and an identity-sensitive field may be redacted. The gate generates admissibility result records and provides admitted records to the governed context bundle assembler.

The governed context bundle assembler deterministically assembles admitted memory atoms and permitted transformed records into a governed context bundle. The governed context bundle may include a bundle identifier, bundle digest, policy snapshot identifier, passenger identity reference, flight identifier, task identifier, timestamp, transformation records, and admissibility results. An execution governance artifact is generated or associated with the governed context bundle.

The constrained execution interface verifies the governed execution package. If the governed context bundle and execution governance artifact are present and valid, the monitoring or reasoning component executes using the governed context bundle as its patient-scoped input. If the governed execution package is absent, invalid, expired, mismatched, or inconsistent with the aviation policy snapshot, execution is denied, delayed, quarantined, or limited to non-patient-specific fallback guidance.

The monitoring or reasoning component may generate an advisory output, alert, telemetry summary, recommendation, documentation draft, or remote monitoring package. The output does not automatically create reliance. Before the output is displayed as actionable medical information, documented, transmitted, forwarded, used for physician coordination, or presented to a remote monitoring center, the system generates or verifies a readiness artifact bound to the governed context bundle and the aviation policy snapshot.

If the readiness artifact is valid for the intended reliance event, the governed connector may allow the downstream action. For example, the system may display a crew-facing advisory, transmit a telemetry summary to a remote monitoring center, or make a documentation-ready record available to an authorized interface. If readiness verification fails, the system may deny, defer, quarantine, or limit the downstream action and may provide a crew status indication or non-patient-specific fallback guidance.

When off-aircraft transmission is permitted, the governed connector transmits the permitted payload through an available communication path, such as satellite, air-to-ground, Wi-Fi, cellular, or another approved link. The payload may include telemetry summaries, alerts, recommendations, readiness artifact information, governed context identifiers, policy identifiers, and audit references. The remote monitoring center verifies the readiness artifact before accepting, storing, displaying, forwarding, documenting, or acting upon the payload.

If the remote monitoring center verifies the readiness artifact, a remote clinician interface may present the permitted information to a remote clinician. If verification fails, the remote monitoring center may reject the payload, quarantine the payload, request retransmission, or record a failed-readiness event. The system may store a receipt-bound governance record linking the transmission authorization decision to the remote delivery, acceptance, rejection, quarantine, or verification result.

In a connectivity-loss example, satellite communication may become unavailable during the medical event. The system may continue to capture telemetry locally, store memory atoms, perform gate evaluation, and generate a governed context bundle. If off-aircraft transmission is not permitted or not possible, the governed connector may buffer the payload and mark transmission as delayed. Later transmission may occur only if freshness, ordering, consent, policy, and readiness conditions remain satisfied. Otherwise, the payload may be retained for audit, transformed, quarantined, or denied for reliance.

In a multiple-passenger example, a second passenger may also require monitoring during the same flight. The system creates or uses a separate patient partition for the second passenger. Candidate memory atoms for the first passenger and second passenger are evaluated separately. Separate governed context bundles, execution governance artifacts, readiness artifacts, transmission records, and audit records are generated. This prevents telemetry or outputs associated with one passenger from being used for the other passenger unless an aviation policy snapshot permits a specific transformed or aggregated use.

In a fail-closed example, a medical device may fail authentication, a readiness artifact may be missing, or a governed connector may be unavailable. The system may deny patient-specific execution, prevent off-aircraft transmission, prevent display as actionable medical information, quarantine the affected payload, or store the record for audit only. Cabin crew may be instructed to follow standard cabin medical procedures while the system records the failure condition and response.

In a fail-quiet example, the system may detect degraded cabin wireless performance, excessive retries, or potential RF coexistence constraints. The system may reduce transmit power, reduce scanning frequency, change communication channel, select another proxy node, buffer telemetry, delay noncritical transmission, or suppress repeated failed alerts. This allows the system to continue operating within cabin constraints without creating unnecessary communication burden or unreliable outputs.

The disclosed architecture provides several technical effects. It converts aircraft-cabin telemetry from unbound device data into governed patient-scoped records. It maintains patient partitioning in a moving, temporary, and connectivity-constrained environment. It preserves provenance through proxy handoff and device changes. It prevents monitoring or reasoning components from using patient-scoped context unless candidate records have been admitted or transformed by a non-bypassable memory gate. It binds execution to a governed context bundle and execution governance artifact. It conditions downstream reliance on readiness artifact verification. It controls off-aircraft egress through a governed connector and supports receiver-side verification by a remote monitoring center.

The architecture may reduce reliance on stale, unauthenticated, misidentified, or unconsented telemetry. It may reduce uncontrolled transmission of medical data from an aircraft cabin. It may improve the quality and traceability of information available to cabin crew, onboard clinicians, remote clinicians, or operator medical support services. It may preserve crew authority and standard emergency procedures while providing a governed technical path for capture, admission, execution, reliance, transmission, remote verification, and audit.

The architecture also supports aircraft-specific constraints. It may be deployed without redesigning the aircraft cabin or connecting to primary avionics buses. It may operate as a cabin medical overlay network, adapt to RF coexistence conditions, buffer telemetry during connectivity loss, fail closed or fail quiet when required conditions are not satisfied, and maintain isolation from aircraft navigation, propulsion, flight-control, and primary avionics functions.

### DETAILED DESCRIPTION OF THE FIGURES

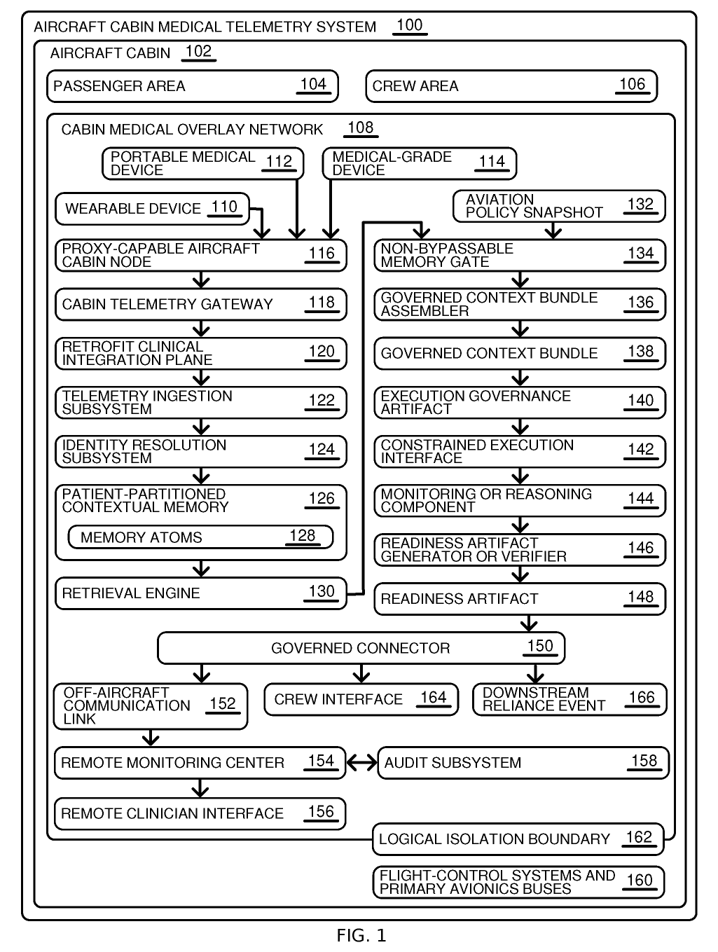

FIG. 1 - Overall Aircraft Cabin Medical Telemetry Architecture

FIG. 1 illustrates an aircraft cabin medical telemetry system 100 configured to acquire medical or physiologic telemetry within an aircraft cabin 102, associate the telemetry with a passenger identity, store the telemetry in patient-partitioned contextual memory, govern access to patient-scoped context before execution of a monitoring or reasoning component, and condition downstream reliance on verification of a readiness artifact.

The aircraft cabin 102 may include a passenger area 104 and a crew area 106. A cabin medical overlay network 108 operates within or in association with the aircraft cabin 102. The cabin medical overlay network 108 may be implemented as a logical and/or physical medical telemetry overlay that is separate from passenger entertainment, general passenger Internet access, flight-control systems, and primary avionics systems. In some embodiments, the cabin medical overlay network 108 is deployed as a retrofit system or temporary emergency-use system without structural modification of the aircraft cabin 102.

One or more wearable devices 110, portable medical devices 112, or medical-grade devices 114 generate telemetry during a flight. The telemetry may include physiologic measurements, device status information, communication state information, alarm state information, or derived values. The devices 110, 112, 114 may communicate with at least one proxy-capable aircraft cabin node 116. The proxy-capable aircraft cabin node 116 may be seat-integrated, crew-carried, passenger-carried, dongle-based, cart-mounted, wall-powered, battery-powered, or temporarily activated during an in-flight medical event.

The proxy-capable aircraft cabin node 116 routes received telemetry to a cabin telemetry gateway 118. The cabin telemetry gateway 118 may buffer, route, normalize, timestamp, authenticate, or otherwise prepare telemetry for processing by a retrofit clinical integration plane 120. The retrofit clinical integration plane 120 provides a controlled acquisition layer for telemetry generated within the aircraft cabin 102. The retrofit clinical integration plane 120 may operate without requiring access to flight-control systems or primary avionics buses.

A telemetry ingestion subsystem 122 receives telemetry from the retrofit clinical integration plane 120. The telemetry ingestion subsystem 122 may normalize heterogeneous telemetry formats, associate telemetry with source and timing information, and generate machine-readable records suitable for governed processing. In some embodiments, the telemetry ingestion subsystem 122 receives raw telemetry, normalized telemetry, derived values, device status records, communication status records, or proxy-node status records.

An identity resolution subsystem 124 associates received telemetry with a passenger identity. The identity resolution subsystem 124 may use one or more of manifest information, seat assignment, consent state, device association, passenger mobile credential, crew verification, emergency token, temporary patient profile, companion confirmation, remote clinician confirmation, or post-event reconciliation. The result of the identity resolution process may be a resolved passenger identity, a confidence state, or a temporary patient partition pending later reconciliation.

A patient-partitioned contextual memory 126 stores telemetry and derived information as memory atoms 128. The patient-partitioned contextual memory 126 maintains patient-scoped separation so that telemetry associated with one passenger is not made available as patient-scoped context for another passenger unless allowed by an applicable policy. Each memory atom 128 may include telemetry or derived contextual information together with provenance metadata, time metadata, device metadata, proxy node metadata, seat metadata, flight metadata, consent metadata, trust metadata, route or jurisdiction metadata, and transformation history.

A retrieval engine 130 selects candidate memory atoms 128 responsive to a monitoring task request. The retrieval engine 130 may select candidate memory atoms 128 based on passenger identity, flight identifier, task type, time window, device type, source trust state, consent state, escalation state, or other retrieval criteria. Selection by the retrieval engine 130 does not by itself make the candidate memory atoms 128 available for execution by a monitoring or reasoning component.

An aviation policy snapshot 132 represents the policy state applicable to the monitoring task, passenger, flight, route, jurisdiction, consent state, crew role, escalation state, connectivity state, or remote monitoring availability. The aviation policy snapshot 132 may encode operator rules, route-dependent privacy rules, jurisdiction-dependent privacy rules, consent rules, retention rules, credentialing constraints, escalation thresholds, crew role permissions, remote clinician permissions, satellite connectivity constraints, emergency-mode rules, or flight-phase-dependent rules.

A non-bypassable memory gate 134 evaluates the candidate memory atoms 128 under the aviation policy snapshot 132 and applicable integrity criteria before a monitoring or reasoning component is allowed to access patient-scoped context. The integrity criteria may include provenance verification, temporal validity evaluation, source trust classification, conflict detection, completeness evaluation, device-authentication status, seat-identity consistency, consent status, route-policy status, or transformation-history evaluation. The non-bypassable memory gate 134 may admit, deny, redact, abstract, attenuate, quarantine, or transform each candidate memory atom 128.

A governed context bundle assembler 136 deterministically assembles admitted memory atoms into a governed context bundle 138. The governed context bundle 138 may include admitted memory atoms, a bundle identifier, a bundle digest, a policy snapshot identifier, a passenger identity reference, a flight identifier, a task identifier, a timestamp, and a record of transformations applied by the non-bypassable memory gate 134. In some embodiments, the governed context bundle 138 is serialized in a canonical form so that downstream components can verify that the same admitted context was used.

An execution governance artifact 140 is associated with the governed context bundle 138. The execution governance artifact 140 may record admissibility outcomes, policy snapshot information, integrity results, bundle identifiers, transformation records, or execution authorization information. A constrained execution interface 142 permits a monitoring or reasoning component 144 to execute only when the governed context bundle 138 and the execution governance artifact 140 are present and valid. If the governed context bundle 138 or the execution governance artifact 140 is absent, expired, mismatched, invalid, or inconsistent with the aviation policy snapshot 132, execution may be denied, delayed, quarantined, or limited to non-patient-specific fallback content.

The monitoring or reasoning component 144 may include a rules-based module, signal processing module, predictive module, AI-assisted case manager, remote monitoring preparation module, alerting module, or other component configured to generate an advisory output, summary, alert, recommendation, or documentation-ready record. The monitoring or reasoning component 144 does not receive unrestricted access to patient-scoped contextual memory. Instead, it receives only the governed context bundle 138 admitted through the non-bypassable memory gate 134.

A readiness artifact generator or verifier 146 generates or verifies a readiness artifact 148. The readiness artifact 148 is bound to the governed context bundle 138 and the aviation policy snapshot 132. The readiness artifact 148 may include a bundle identifier, aviation policy snapshot identifier, time reference, verification digest, digital signature, nonce, evaluator identifier, execution certificate, readiness certificate, policy-verification result, or transmission authorization record.

A governed connector 150 controls downstream reliance and off-aircraft egress. The governed connector 150 may condition a downstream reliance event 166 on verification of the readiness artifact 148. The downstream reliance event 166 may include crew notification, rendering, display, documentation, storage as actionable medical information, physician coordination, remote monitoring center acceptance, forwarding, transmission, or diversion-related decision support. In some embodiments, the governed connector 150 permits an off-aircraft communication link 152 to transmit telemetry, alerts, recommendations, or summaries only after verification of the readiness artifact 148.

The off-aircraft communication link 152 may include a satellite link, air-to-ground link, Wi-Fi link, cellular link, or other communication path. A remote monitoring center 154 may receive a transmitted payload and may independently verify the readiness artifact 148 before accepting, storing, displaying, forwarding, documenting, or acting upon the payload. A remote clinician interface 156 may present verified information to a remote clinician only after the required receiver-side verification is satisfied. If verification fails, the payload may be rejected, quarantined, held for retransmission, or recorded as a failed-readiness event.

An audit subsystem 158 records evidence of system operation. The audit subsystem 158 may store telemetry ingestion records, identity resolution records, memory atom records, policy snapshot identifiers, admissibility results, governed context bundle identifiers, execution governance artifacts, readiness artifacts, transmission records, delivery receipts, remote verification outcomes, failed-readiness events, proxy handoff records, and downstream reliance records. The audit subsystem 158 may support reconstruction of the path from telemetry capture to downstream reliance.

The aircraft cabin medical telemetry system 100 is separated from flight-control systems and primary avionics buses 160 by a logical isolation boundary 162. The logical isolation boundary 162 indicates that the system 100 does not command, alter, or interfere with aircraft navigation, propulsion, flight-control, or primary avionics functions. In some embodiments, the system 100 may receive or use cabin connectivity resources through mediated interfaces while maintaining non-interference with aircraft operational systems.

A crew interface 164 may provide crew-facing information, readiness state, transmission state, advisory output, fallback status, or escalation prompts. The crew interface 164 may display only information permitted by the applicable aviation policy snapshot 132 and readiness verification state. The crew interface 164 preserves crew authority and standard cabin medical procedures.

Accordingly, FIG. 1 shows a controlled technical path in which aircraft-cabin telemetry is acquired through proxy-capable cabin infrastructure, associated with a passenger identity, stored in patient-partitioned contextual memory, admitted through a non-bypassable memory gate, assembled into a governed context bundle, used only through a constrained execution interface, and relied upon only after readiness artifact verification.

REFERENCE LIST 100 — AIRCRAFT CABIN MEDICAL TELEMETRY SYSTEM 102 — AIRCRAFT CABIN 104 — PASSENGER AREA

106 — CREW AREA 108 — CABIN MEDICAL OVERLAY NETWORK 110 — WEARABLE DEVICE 112 — PORTABLE MEDICAL DEVICE 114 — MEDICAL-GRADE DEVICE 116 — PROXY-CAPABLE AIRCRAFT CABIN NODE 118 — CABIN TELEMETRY GATEWAY 120 — RETROFIT CLINICAL INTEGRATION PLANE 122 — TELEMETRY INGESTION SUBSYSTEM 124 — IDENTITY RESOLUTION SUBSYSTEM 126 — PATIENT-PARTITIONED CONTEXTUAL MEMORY 128 — MEMORY ATOMS 130 — RETRIEVAL ENGINE 132 — AVIATION POLICY SNAPSHOT 134 — NON-BYPASSABLE MEMORY GATE 136 — GOVERNED CONTEXT BUNDLE ASSEMBLER 138 — GOVERNED CONTEXT BUNDLE 140 — EXECUTION GOVERNANCE ARTIFACT 142 — CONSTRAINED EXECUTION INTERFACE 144 — MONITORING OR REASONING COMPONENT 146 — READINESS ARTIFACT GENERATOR OR VERIFIER 148 — READINESS ARTIFACT 150 — GOVERNED CONNECTOR 152 — OFF-AIRCRAFT COMMUNICATION LINK 154 — REMOTE MONITORING CENTER 156 — REMOTE CLINICIAN INTERFACE 158 — AUDIT SUBSYSTEM 160 — FLIGHT-CONTROL SYSTEMS AND PRIMARY AVIONICS BUSES 162 — LOGICAL ISOLATION BOUNDARY 164 — CREW INTERFACE 166 — DOWNSTREAM RELIANCE EVENT

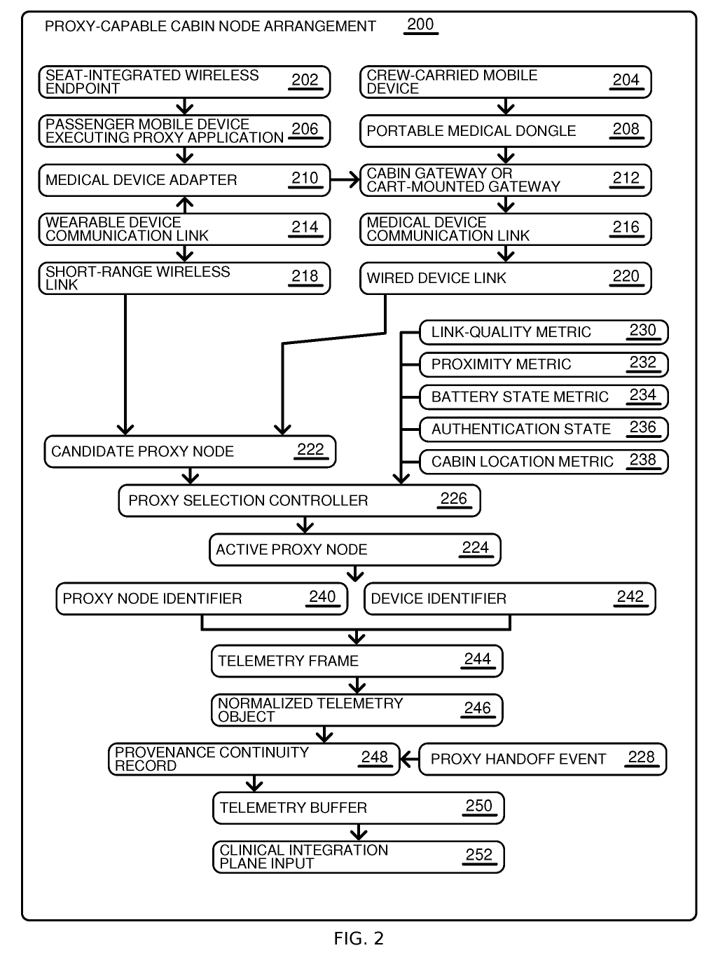

FIG. 2 - Proxy-Capable Cabin Nodes and Telemetry Capture

FIG. 2 illustrates a proxy-capable cabin node arrangement 200 configured to receive telemetry from wearable devices, portable medical devices, medical-grade devices, crew-carried devices, passenger-carried devices, dongles, adapters, or other aircraft-cabin telemetry sources and route the telemetry into the clinical integration plane for governed processing.

The proxy-capable cabin node arrangement 200 may include a seat-integrated wireless endpoint 202. The seat-integrated wireless endpoint 202 may be located at, under, adjacent to, or otherwise associated with a passenger seat, seat row, cabin zone, service panel, seat power interface, seat electronics module, or other cabin structure. In some embodiments, the seat-integrated wireless endpoint 202 communicates with a wearable device worn by a passenger assigned to or located near the associated seat.

The proxy-capable cabin node arrangement 200 may also include a crew-carried mobile device 204. The crew-carried mobile device 204 may be a tablet, smartphone, handheld terminal, medical kit device, crew communication device, or other portable device carried or operated by cabin crew. The crew-carried mobile device 204 may operate as a proxy node during an in-flight medical event, including when crew deploys a portable medical device, obtains a measurement, verifies a passenger identity, or assists with remote medical support.

A passenger mobile device executing a proxy application 206 may also serve as a proxy-capable cabin node. The proxy application may receive telemetry from a wearable device or medical device associated with the passenger and may forward telemetry to the cabin telemetry gateway or clinical integration plane only when device authorization, consent state, communication state, and applicable aviation policy conditions permit such forwarding. In some embodiments, the passenger mobile device 206 is limited to proxy operation and does not itself make clinical reliance decisions.

The proxy-capable cabin node arrangement 200 may include a portable medical dongle 208. The portable medical dongle 208 may be connected to or associated with a wearable device, portable medical device, crew device, passenger device, emergency medical kit, seat power interface, or service interface. The portable medical dongle 208 may provide device identification, signal conversion, local buffering, wireless communication, wired communication, or authentication functions.

A medical device adapter 210 may provide an interface between a medical device and the aircraft cabin medical telemetry system. The medical device adapter 210 may support wired serial communication, USB communication, Ethernet communication, wireless communication, optical communication, near-field communication, or another physical or logical communication path. In some embodiments, the medical device adapter 210 converts device-specific telemetry into a format suitable for ingestion by the clinical integration plane.

A cabin gateway or cart-mounted gateway 212 may aggregate telemetry from one or more proxy-capable cabin nodes. The cabin gateway or cart-mounted gateway 212 may be installed in the aircraft cabin, carried on a medical cart, included in an emergency medical kit, mounted at a galley or service area, or otherwise positioned within the aircraft cabin. The gateway 212 may route telemetry to the cabin medical overlay network, apply local filtering or buffering, and forward telemetry for governed processing.

A wearable device communication link 214 may be established between a wearable device and one or more proxy-capable cabin nodes. The wearable device communication link 214 may carry physiologic measurements, device status, pairing information, device identity, time information, signal quality information, or other wearable-device data. A medical device communication link 216 may be established between a portable or medical-grade device and one or more proxy-capable cabin nodes. The medical device communication link 216 may carry measurements, device state, alarm state, operating state, connection state, or other medical-device data.

The communication paths may include a short-range wireless link 218. The short-range wireless link 218 may include Bluetooth, Bluetooth Low Energy, Wi-Fi, near-field communication, ultra-wideband, optical wireless communication, or another short-range communication protocol. The communication paths may also include a wired device link 220. The wired device link 220 may include USB, serial, Ethernet, proprietary cable, medical-device adapter connection, seat power data interface, or another wired communication path.

During operation, more than one proxy-capable cabin node may be available to receive telemetry from a wearable device or medical device. A candidate proxy node 222 is a node that is available for possible proxy operation. An active proxy node 224 is the node selected to receive or route telemetry for a particular device, passenger, seat location, cabin zone, or medical event. The active proxy node 224 may change during the flight.

A proxy selection controller 226 determines which candidate proxy node 222 should serve as the active proxy node 224. The proxy selection controller 226 may be implemented in a cabin node, cabin gateway, crew device, edge processor, clinical integration plane, or distributed combination of those components. The proxy selection controller 226 may evaluate one or more operating metrics before selecting or reselecting the active proxy node 224.

A proxy handoff event 228 may occur when the active proxy node 224 changes from a first proxy-capable cabin node to a second proxy-capable cabin node. The proxy handoff event 228 may be triggered by passenger movement, device movement, node unavailability, degraded communication, authentication failure, battery depletion, cabin-location change, crew intervention, medical-device deployment, or policy change. The proxy handoff event 228 may be recorded so that telemetry provenance remains continuous even when the proxy path changes.

The proxy selection controller 226 may use a link-quality metric 230. The link-quality metric 230 may include signal strength, packet loss, latency, error rate, retry count, channel quality, interference condition, connection stability, or another metric indicating communication quality. The proxy selection controller 226 may also use a proximity metric 232. The proximity metric 232 may indicate physical proximity between a device and a proxy node, between a passenger and a seat location, between a crew device and a passenger, or between a medical device and a cabin gateway.

A battery state metric 234 may indicate battery level, charging state, estimated remaining operating time, power source availability, or power stability of a candidate node or connected device. An authentication state 236 may indicate whether a device, node, dongle, adapter, mobile application, gateway, or communication path has been authenticated, authorized, enrolled, or revoked. A cabin location metric 238 may indicate seat location, cabin zone, galley location, aisle location, lavatory proximity, crew area, medical cart position, or other aircraft-cabin location information.

When telemetry is received or routed, the system may associate the telemetry with a proxy node identifier 240. The proxy node identifier 240 may identify the active proxy node 224, candidate proxy node 222, seat-integrated wireless endpoint 202, crew-carried mobile device 204, passenger mobile device 206, portable medical dongle 208, medical device adapter 210, cabin gateway 212, or other node involved in telemetry capture. The system may also associate the telemetry with a device identifier 242. The device identifier 242 may identify the wearable device, portable medical device, medical-grade device, dongle, adapter, or other telemetry source.

A telemetry frame 244 may be received through the active proxy node 224. The telemetry frame 244 may include raw measurement data, device status, alarm state, communication state, source time, device time, sequence number, signal-quality information, or original payload content. The telemetry frame 244 may be converted into a normalized telemetry object 246. The normalized telemetry object 246 may include standardized fields for device identity, proxy node identity, passenger or seat association, flight association, event time, receipt time, measurement value, units, data quality, source trust state, and provenance information.

When a proxy handoff event 228 occurs, the system may generate or update a provenance continuity record 248. The provenance continuity record 248 may identify a prior active proxy node, a newly selected active proxy node, the triggering condition for handoff, the time of handoff, associated device identifiers, associated passenger or seat identifiers, and any telemetry sequence information needed to determine whether telemetry was continuous, delayed, duplicated, or lost. The provenance continuity record 248 may later be stored in, or referenced by, memory atoms and audit records.

A telemetry buffer 250 may temporarily store telemetry frames 244, normalized telemetry objects 246, proxy handoff records, or device status records. The telemetry buffer 250 may be located in a cabin node, mobile device, dongle, gateway, edge processor, or clinical integration plane. Buffering may be used when link quality is degraded, the cabin gateway is unavailable, satellite connectivity is unavailable, authentication is pending, policy evaluation is delayed, or a proxy handoff is in progress.

The clinical integration plane input 252 receives normalized telemetry objects 246, provenance continuity records 248, telemetry buffer output, and related proxy-state information. The clinical integration plane input 252 forwards the information into the telemetry ingestion subsystem and subsequent governance components described with respect to FIG. 1. In this manner, FIG. 2 shows how aircraft-cabin telemetry may be acquired through flexible proxy-capable nodes while preserving device identity, proxy identity, communication state, location context, and provenance continuity before the telemetry is used in patient-partitioned contextual memory.

REFERENCE LIST 200 — PROXY-CAPABLE CABIN NODE ARRANGEMENT 202 — SEAT-INTEGRATED WIRELESS ENDPOINT 204 — CREW-CARRIED MOBILE DEVICE 206 — PASSENGER MOBILE DEVICE EXECUTING PROXY APPLICATION 208 — PORTABLE MEDICAL DONGLE 210 — MEDICAL DEVICE ADAPTER 212 — CABIN GATEWAY OR CART-MOUNTED GATEWAY 214 — WEARABLE DEVICE COMMUNICATION LINK 216 — MEDICAL DEVICE COMMUNICATION LINK 218 — SHORT-RANGE WIRELESS LINK 220 — WIRED DEVICE LINK 222 — CANDIDATE PROXY NODE 224 — ACTIVE PROXY NODE 226 — PROXY SELECTION CONTROLLER 228 — PROXY HANDOFF EVENT 230 — LINK-QUALITY METRIC 232 — PROXIMITY METRIC 234 — BATTERY STATE METRIC 236 — AUTHENTICATION STATE 238 — CABIN LOCATION METRIC 240 — PROXY NODE IDENTIFIER 242 — DEVICE IDENTIFIER 244 — TELEMETRY FRAME 246 — NORMALIZED TELEMETRY OBJECT 248 — PROVENANCE CONTINUITY RECORD 250 — TELEMETRY BUFFER 252 — CLINICAL INTEGRATION PLANE INPUT

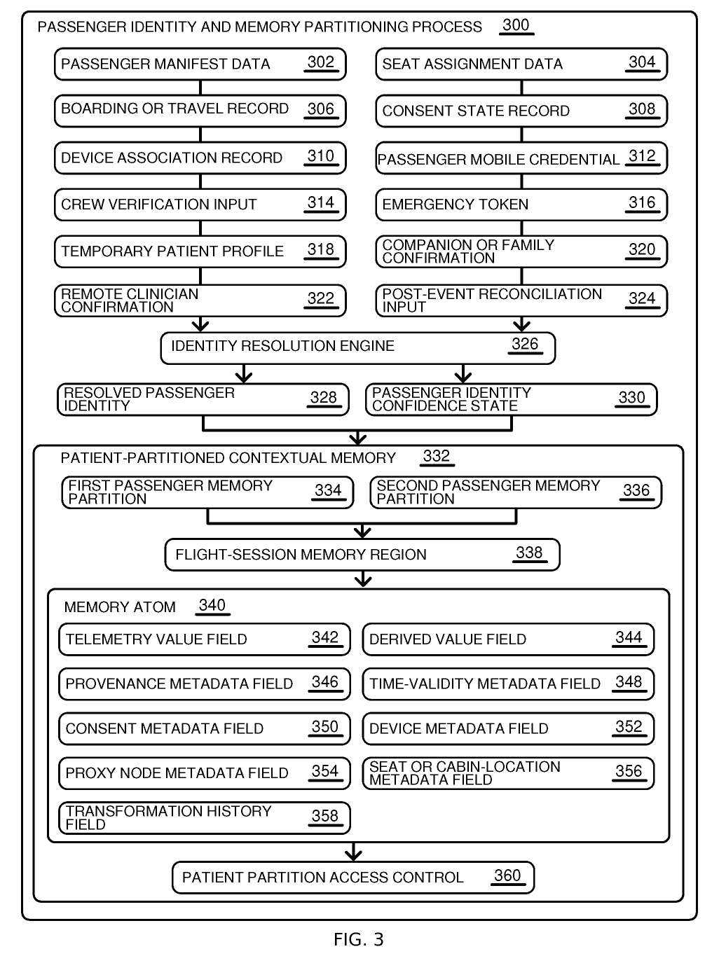

FIG. 3 - Passenger Identity Resolution and Patient-Partitioned Memory

FIG. 3 illustrates a passenger identity and memory partitioning process 300 configured to associate aircraft-cabin telemetry with a passenger identity and to store the telemetry in a patient-partitioned contextual memory before the telemetry is used by a monitoring or reasoning component.

The passenger identity and memory partitioning process 300 may receive passenger manifest data 302. The passenger manifest data 302 may include passenger name, passenger identifier, booking identifier, flight identifier, itinerary information, or other data available from an airline, operator, reservation system, boarding system, or crew system. The passenger manifest data 302 may be used alone or with other inputs to resolve or confirm the identity of a passenger involved in an in-flight medical event.

Seat assignment data 304 may also be used. The seat assignment data 304 may identify an assigned seat, current seat, seat row, cabin zone, temporary relocation, or crew-observed passenger location. Seat assignment data 304 may be derived from a reservation record, boarding record, crew entry, seat-integrated node, cabin map, or post-event update. In some embodiments, seat assignment data 304 provides an initial association between a passenger identity and telemetry received through a seat-associated proxy-capable cabin node.

Boarding or travel record 306 may provide additional identity or context information. The boarding or travel record 306 may include boarding status, boarding time, travel document reference, itinerary segment, passenger status, special service code, emergency contact reference, or other travel-related information. The boarding or travel record 306 may be used to confirm that a passenger associated with a telemetry source is present on the flight.

A consent state record 308 may identify whether the passenger has granted, denied, limited, revoked, or not yet provided consent for telemetry capture, processing, transmission, remote monitoring, documentation, or other downstream use. The consent state record 308 may be created through a passenger mobile application, crew interface, seat interface, emergency workflow, travel record, biometric verification process, or other consent capture process. The consent state record 308 may be stored as contextual information and may be evaluated under an aviation policy snapshot.

A device association record 310 may associate a wearable device, portable medical device, dongle, adapter, passenger mobile device, or other telemetry source with a passenger. The device association record 310 may be created by pairing, scanning, device enrollment, prior registration, passenger mobile credential, crew confirmation, emergency association, or post-event reconciliation. The device association record 310 may include a device identifier, device type, pairing time, association confidence, and the source of the association.

A passenger mobile credential 312 may be used as an identity input. The passenger mobile credential 312 may be provided by an airline application, wallet credential, health application, boarding pass, emergency medical credential, wearable-device account, or other mobile credential associated with the passenger. The passenger mobile credential 312 may be verified locally, through cabin connectivity, through a crew device, or through a remote service when available.

Crew verification input 314 may be provided by a flight attendant, medical crew member, captain, onboard clinician, or other authorized person. The crew verification input 314 may confirm a passenger identity, seat location, consent status, device association, temporary patient profile, or observed condition. In some embodiments, crew verification input 314 is stored with a user identity, role, timestamp, and confidence value.

An emergency token 316 may be used when ordinary identity sources are unavailable, incomplete, or unreliable. The emergency token 316 may be generated by the system, by a crew interface, by a mobile device, by a medical kit device, by a remote monitoring center, or by another authorized component. The emergency token 316 may permit telemetry to be associated with an emergency patient partition until a passenger identity is later confirmed or reconciled.

A temporary patient profile 318 may be created during an in-flight medical event. The temporary patient profile 318 may include a temporary patient identifier, seat or cabin location, observed age range, observed sex if relevant and permitted, crew notes, device association, consent status, and event time. The temporary patient profile 318 allows telemetry to be stored in a patient-partitioned manner even when a final passenger identity is not yet available.

Companion or family confirmation 320 may provide identity information when the passenger cannot self-identify or when manifest or seat information is uncertain. The confirmation 320 may be received through a crew interface and may be stored with source, role, time, and confidence information. Remote clinician confirmation 322 may also provide identity or association information when a remote medical support service participates in the event.

Post-event reconciliation input 324 may be used after the in-flight medical event or after connectivity is restored. The post-event reconciliation input 324 may reconcile temporary patient profiles, emergency tokens, device association records, crew notes, manifest records, seat records, and remote monitoring records. Post-event reconciliation may update identity confidence, correct a prior association, merge or separate patient partitions, or mark records as unresolved while preserving the earlier provenance.

An identity resolution engine 326 receives one or more of the identity-related inputs. The identity resolution engine 326 may compare, rank, validate, or reconcile identity signals. The identity resolution engine 326 may use source priority rules, aviation policy rules, confidence thresholds, matching rules, time windows, seat-location consistency, device association consistency, crew role permissions, consent rules, and emergency-mode rules. The identity resolution engine 326 may create a resolved passenger identity 328 or may create or maintain a temporary identity state pending further confirmation.

A passenger identity confidence state 330 may be assigned to the resolved passenger identity 328 or to a temporary patient profile. The passenger identity confidence state 330 may indicate confirmed identity, probable identity, temporary identity, anonymous emergency partition, conflicting identity, pending reconciliation, or unresolved identity. The passenger identity confidence state 330 may affect admissibility of memory atoms, transmission, display, documentation, remote monitoring acceptance, retention, or downstream reliance.

A patient-partitioned contextual memory 332 stores telemetry and related contextual information in patient-specific partitions. In the example shown in FIG. 3, the patient-partitioned contextual memory 332 includes a first passenger memory partition 334 and a second passenger memory partition 336. Additional partitions may be created for additional passengers or temporary emergency patient profiles. Each partition may be logically separated from other partitions so that patient-scoped context for one passenger is not used for another passenger unless allowed by an applicable aviation policy snapshot.

A flight-session memory region 338 may be associated with a particular flight, flight segment, passenger identity, temporary profile, or emergency event. The flight-session memory region 338 may store records for the duration of the flight or for another policy-defined retention period. A flight-session memory region 338 may be keyed by flight identifier, passenger identity reference, temporary patient profile identifier, seat identifier, device identifier, or other partitioning information.

Telemetry and derived values may be stored as memory atoms 340. A memory atom 340 is a structured contextual record that includes patient-scoped information and associated governance metadata. A memory atom 340 may include a telemetry value field 342 containing a measured value, waveform-derived value, device state, alarm state, communication state, or raw telemetry element. A derived value field 344 may store a calculated trend, summary, risk indicator, quality score, confidence score, or other value derived from one or more telemetry records or observations.

A provenance metadata field 346 may identify the origin and path of the data. The provenance metadata field 346 may include source device, source system, proxy node, crew user, passenger device, gateway, communication link, ingestion subsystem, transformation step, or other source information. A time-validity metadata field 348 may include event time, device time, receipt time, processing time, validity interval, expiration time, or stale-data state.

A consent metadata field 350 may store consent state, consent source, consent time, revocation state, emergency override state, or consent limitation associated with the memory atom 340. A device metadata field 352 may include device identifier, device type, device authentication state, device trust state, device quality state, device battery state, device firmware state, or device communication state. A proxy node metadata field 354 may identify the active proxy node, prior proxy node, candidate proxy node, proxy handoff event, proxy trust state, or proxy communication metrics.

A seat or cabin-location metadata field 356 may identify assigned seat, current seat, cabin zone, crew-observed location, seat node, galley location, aisle location, or other location context. A transformation history field 358 may identify normalization, redaction, abstraction, attenuation, quarantine, filtering, unit conversion, confidence adjustment, or other processing applied to the record. The transformation history field 358 may support later gate evaluation and audit reconstruction.

Patient partition access control 360 controls access to the patient-partitioned contextual memory 332 and its memory atoms 340. The patient partition access control 360 may prevent a retrieval engine, monitoring component, reasoning component, crew interface, remote monitoring center, or documentation system from accessing patient-scoped records outside the permitted passenger partition. The patient partition access control 360 may operate with the aviation policy snapshot, identity confidence state, consent state, role state, and task context.

Accordingly, FIG. 3 shows that telemetry received within an aircraft cabin is not treated as free-floating device data. The telemetry is associated with passenger identity context, confidence state, consent state, device context, proxy context, time context, and location context, and is stored as memory atoms in patient-partitioned contextual memory for later governed evaluation.

REFERENCE LIST 300 — PASSENGER IDENTITY AND MEMORY PARTITIONING PROCESS 302 — PASSENGER MANIFEST DATA 304 — SEAT ASSIGNMENT DATA 306 — BOARDING OR TRAVEL RECORD 308 — CONSENT STATE RECORD 310 — DEVICE ASSOCIATION RECORD 312 — PASSENGER MOBILE CREDENTIAL 314 — CREW VERIFICATION INPUT 316 — EMERGENCY TOKEN 318 — TEMPORARY PATIENT PROFILE 320 — COMPANION OR FAMILY CONFIRMATION 322 — REMOTE CLINICIAN CONFIRMATION 324 — POST-EVENT RECONCILIATION INPUT 326 — IDENTITY RESOLUTION ENGINE 328 — RESOLVED PASSENGER IDENTITY 330 — PASSENGER IDENTITY CONFIDENCE STATE 332 — PATIENT-PARTITIONED CONTEXTUAL MEMORY 334 — FIRST PASSENGER MEMORY PARTITION 336 — SECOND PASSENGER MEMORY PARTITION 338 — FLIGHT-SESSION MEMORY REGION 340 — MEMORY ATOM 342 — TELEMETRY VALUE FIELD 344 — DERIVED VALUE FIELD 346 — PROVENANCE METADATA FIELD 348 — TIME-VALIDITY METADATA FIELD 350 — CONSENT METADATA FIELD 352 — DEVICE METADATA FIELD 354 — PROXY NODE METADATA FIELD 356 — SEAT OR CABIN-LOCATION METADATA FIELD 358 — TRANSFORMATION HISTORY FIELD 360 — PATIENT PARTITION ACCESS CONTROL

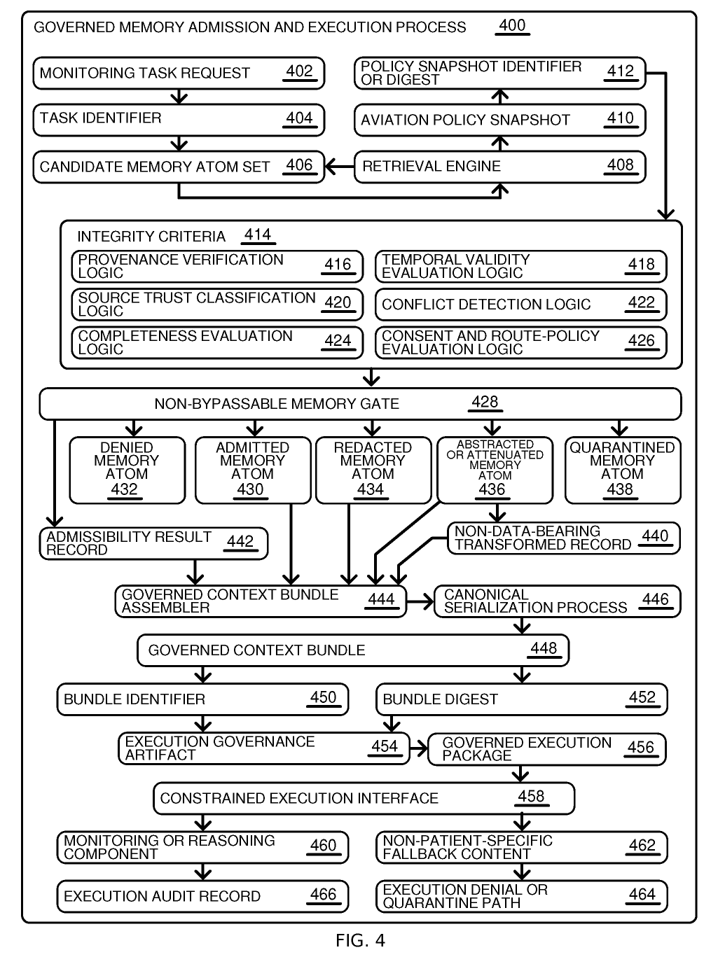

FIG. 4 - Memory Gate, Governed Context Bundle, and Constrained Execution

FIG. 4 illustrates a governed memory admission and execution process 400 configured to control whether patient-scoped aircraft-cabin telemetry may be used by a monitoring or reasoning component. The governed memory admission and execution process 400 operates after telemetry has been associated with a passenger identity and stored as memory atoms in patient-partitioned contextual memory.

A monitoring task request 402 initiates the process. The monitoring task request 402 may be generated by a crew interface, remote monitoring center, monitoring module, escalation workflow, rule-based trigger, AI-assisted case manager, medical kit device, cabin gateway, or another authorized component. The monitoring task request 402 may request generation of an alert, summary, advisory output, telemetry review, remote monitoring package, documentation record, or other patient-scoped output.

A task identifier 404 may be associated with the monitoring task request 402. The task identifier 404 may identify the type of monitoring operation requested, the passenger identity or temporary patient profile involved, the flight or flight segment, the requesting component, the requesting role, the time of request, and the policy context applicable to the request. The task identifier 404 may be stored in audit records and may be included in downstream governance artifacts.

A candidate memory atom set 406 is selected for the monitoring task request 402. The candidate memory atom set 406 may include memory atoms corresponding to telemetry values, derived values, device status, proxy-node state, crew observations, consent events, escalation events, remote clinician notes, transmission receipts, or prior readiness verification outcomes. The candidate memory atom set 406 may be selected from a patient-specific partition of contextual memory.

A retrieval engine 408 retrieves the candidate memory atom set 406. The retrieval engine 408 may select candidate records using passenger identity, temporary patient profile, task identifier, flight identifier, seat identifier, device identifier, time window, source trust state, escalation state, consent state, or other retrieval criteria. Retrieval by the retrieval engine 408 does not itself authorize use of the candidate memory atom set 406 by a monitoring or reasoning component.

An aviation policy snapshot 410 provides the policy state used to evaluate the candidate memory atom set 406. The aviation policy snapshot 410 may include airline operator rules, jurisdictional privacy rules, route-dependent rules, consent rules, credentialing rules, retention rules, crew role permissions, remote clinician permissions, connectivity rules, emergency-mode rules, flight-phase rules, and escalation rules. The aviation policy snapshot 410 may be selected or updated based on route, jurisdiction, flight phase, connectivity state, passenger consent state, crew role, remote monitoring availability, or emergency status.

A policy snapshot identifier or digest 412 identifies the aviation policy snapshot 410. The policy snapshot identifier or digest 412 may be included in the governed context bundle, execution governance artifact, readiness artifact, audit record, transmission authorization record, or receipt-bound governance record. Binding downstream artifacts to the policy snapshot identifier or digest 412 allows later verification of which policy state was used to admit, transform, deny, or rely upon patient-scoped information.

Integrity criteria 414 are applied to the candidate memory atom set 406. The integrity criteria 414 may include one or more machine-evaluable checks used to determine whether a memory atom may be admitted, denied, transformed, or otherwise controlled before execution. The integrity criteria 414 may be defined by the aviation policy snapshot 410, by system configuration, by operator policy, by emergency-mode rules, or by remote monitoring requirements.

Provenance verification logic 416 may verify the source and handling path of a candidate memory atom. The provenance verification logic 416 may check source device identity, proxy node identity, cabin gateway identity, crew user identity, ingestion path, transformation history, handoff records, communication path, or trust classification. Temporal validity evaluation logic 418 may determine whether a candidate memory atom is current, stale, out-of-order, superseded, expired, or outside a valid time window for the monitoring task request 402.

Source trust classification logic 420 may classify the source of a candidate memory atom. The source trust classification logic 420 may distinguish medical-grade device data, wearable-device data, passenger-device data, crew-entered observations, remote clinician notes, derived values, buffered data, replayed data, unauthenticated data, or audit-only data. Conflict detection logic 422 may detect inconsistency between candidate memory atoms, between a memory atom and identity data, between telemetry and seat location, between wearable data and medical-grade device data, or between consent state and proposed use.

Completeness evaluation logic 424 may determine whether required supporting information is present. The completeness evaluation logic 424 may check for required passenger identity confidence, consent state, device identifier, event time, proxy node identifier, seat association, policy snapshot identifier, communication state, or other supporting fields. Consent and route-policy evaluation logic 426 may determine whether the candidate memory atom may be used for the requested monitoring task under the passenger consent state, route-specific rules, jurisdiction-specific rules, crew role, remote clinician authorization, retention rule, or emergency-mode condition.

A non-bypassable memory gate 428 evaluates the candidate memory atom set 406 using the aviation policy snapshot 410 and integrity criteria 414. The non-bypassable memory gate 428 prevents patient-scoped contextual input from being supplied to a monitoring or reasoning component unless the candidate memory atoms have been admitted or transformed according to the applicable policy and integrity criteria. The non-bypassable memory gate 428 may be implemented as a policy enforcement point, validation controller, admission controller, access-control service, context firewall, execution precondition service, policy filter, trust broker, or equivalent control function.

The non-bypassable memory gate 428 may classify a candidate memory atom as an admitted memory atom 430 when the record satisfies applicable policy and integrity criteria. A candidate memory atom may be classified as a denied memory atom 432 when use of the record is not permitted. A candidate memory atom may be classified as a redacted memory atom 434 when a portion of the record is removed or masked before use. A candidate memory atom may be classified as an abstracted or attenuated memory atom 436 when a more limited representation is permitted instead of the full original record. A candidate memory atom may be classified as a quarantined memory atom 438 when the record is retained but not used for execution or downstream reliance unless further review or later policy conditions permit use. A candidate memory atom may be converted into a non-data-bearing transformed record 440 when the policy permits retaining a marker, denial reason, or structural placeholder without exposing patient-scoped data.

An admissibility result record 442 may be generated for each evaluated memory atom or for the candidate memory atom set 406. The admissibility result record 442 may include a memory atom identifier, task identifier, passenger identity reference, policy snapshot identifier or digest, integrity criteria applied, admissibility state, transformation applied, denial reason, quarantine reason, evaluator identifier, time of evaluation, and supporting rule identifiers. The admissibility result record 442 may be stored in an audit subsystem and may be referenced by execution or readiness artifacts.

A governed context bundle assembler 444 receives the admitted memory atoms 430 and any permitted redacted, abstracted, attenuated, or non-data-bearing transformed records. The governed context bundle assembler 444 excludes denied memory atoms 432 and quarantined memory atoms 438 unless a later policy-authorized step changes their status. The governed context bundle assembler 444 may operate deterministically so that the same admitted inputs and policy state produce the same or verifiable bundle structure.

A canonical serialization process 446 may serialize the permitted records in a defined order and format. The canonical serialization process 446 may use sorted identifiers, fixed field ordering, normalized units, standardized timestamps, transformation records, policy identifiers, and deterministic encoding. The canonical serialization process 446 may support creation of a digest or signature that allows later verification of the governed context used for execution.

A governed context bundle 448 is produced by the governed context bundle assembler 444. The governed context bundle 448 may be the only patient-scoped contextual input made available to the monitoring or reasoning component for the requested task. The governed context bundle 448 may include admitted memory atoms, transformed records, passenger identity reference, flight identifier, task identifier, policy snapshot identifier, timestamp, transformation records, and admissibility results.

A bundle identifier 450 may identify the governed context bundle 448. A bundle digest 452 may be computed over the canonical serialization of the governed context bundle 448. The bundle identifier 450 and bundle digest 452 may be included in an execution governance artifact, readiness artifact, transmission authorization record, remote verification record, receipt-bound governance record, or audit record.

An execution governance artifact 454 is generated or associated with the governed context bundle 448. The execution governance artifact 454 may include the bundle identifier 450, bundle digest 452, policy snapshot identifier or digest 412, task identifier 404, admissibility result records 442, execution authorization state, expiration time, evaluator identifier, signature, or other verification data. The execution governance artifact 454 provides a machine-verifiable basis for determining whether execution may proceed.

A governed execution package 456 includes the governed context bundle 448 and the execution governance artifact 454. In some embodiments, the governed execution package 456 is required before any patient-specific execution occurs. The governed execution package 456 may be checked for expiration, signature validity, bundle-policy consistency, task consistency, passenger identity consistency, and integrity of the governed context bundle 448.

A constrained execution interface 458 receives the governed execution package 456. The constrained execution interface 458 controls invocation of a monitoring or reasoning component 460. The constrained execution interface 458 may deny access to patient-partitioned contextual memory, raw telemetry, denied records, quarantined records, or unadmitted patient-scoped data outside the governed context bundle 448. The constrained execution interface 458 therefore enforces the rule that the monitoring or reasoning component 460 executes only on governed context.

The monitoring or reasoning component 460 may include a rule engine, signal-processing module, predictive model, AI-assisted case manager, alerting module, summary generator, remote monitoring preparation module, or documentation-preparation module. The monitoring or reasoning component 460 may produce an advisory output, alert, recommendation, summary, trend, classification, documentation draft, or transmission payload. The output remains subject to later readiness-conditioned reliance and does not automatically cause transmission, display, documentation, or crew reliance.

If the governed execution package 456 is absent, invalid, expired, mismatched, or inconsistent with the aviation policy snapshot 410, the constrained execution interface 458 may provide non-patient-specific fallback content 462 instead of patient-specific output. The non-patient-specific fallback content 462 may include standard emergency procedure prompts, general crew workflow guidance, non-patient-specific checklist information, or instructions to obtain additional verification.

An execution denial or quarantine path 464 may be invoked when execution cannot proceed. The execution denial or quarantine path 464 may deny execution, delay execution, quarantine the task, request additional identity confirmation, request updated consent, request device authentication, request policy update, or route the matter for review. An execution audit record 466 may be stored to record the monitoring task request 402, task identifier 404, policy snapshot identifier or digest 412, admissibility results, governed context bundle status, execution governance artifact status, execution decision, denial or quarantine reason, and time of decision.

Accordingly, FIG. 4 shows that the disclosed architecture does not merely retrieve aircraft-cabin telemetry and provide it to a monitoring component. Instead, the architecture evaluates candidate memory atoms under an aviation policy snapshot, creates an auditable admissibility result, assembles admitted records into a governed context bundle, binds the bundle to an execution governance artifact, and permits patient-specific execution only through a constrained execution interface.

### REFERENCE LIST

400 — GOVERNED MEMORY ADMISSION AND EXECUTION PROCESS 402 — MONITORING TASK REQUEST 404 — TASK IDENTIFIER 406 — CANDIDATE MEMORY ATOM SET 408 — RETRIEVAL ENGINE 410 — AVIATION POLICY SNAPSHOT 412 — POLICY SNAPSHOT IDENTIFIER OR DIGEST 414 — INTEGRITY CRITERIA 416 — PROVENANCE VERIFICATION LOGIC 418 — TEMPORAL VALIDITY EVALUATION LOGIC 420 — SOURCE TRUST CLASSIFICATION LOGIC 422 — CONFLICT DETECTION LOGIC 424 — COMPLETENESS EVALUATION LOGIC 426 — CONSENT AND ROUTE-POLICY EVALUATION LOGIC 428 — NON-BYPASSABLE MEMORY GATE 430 — ADMITTED MEMORY ATOM 432 — DENIED MEMORY ATOM 434 — REDACTED MEMORY ATOM 436 — ABSTRACTED OR ATTENUATED MEMORY ATOM 438 — QUARANTINED MEMORY ATOM 440 — NON-DATA-BEARING TRANSFORMED RECORD 442 — ADMISSIBILITY RESULT RECORD 444 — GOVERNED CONTEXT BUNDLE ASSEMBLER 446 — CANONICAL SERIALIZATION PROCESS 448 — GOVERNED CONTEXT BUNDLE 450 — BUNDLE IDENTIFIER 452 — BUNDLE DIGEST 454 — EXECUTION GOVERNANCE ARTIFACT 456 — GOVERNED EXECUTION PACKAGE 458 — CONSTRAINED EXECUTION INTERFACE 460 — MONITORING OR REASONING COMPONENT 462 — NON-PATIENT-SPECIFIC FALLBACK CONTENT 464 — EXECUTION DENIAL OR QUARANTINE PATH 466 — EXECUTION AUDIT RECORD

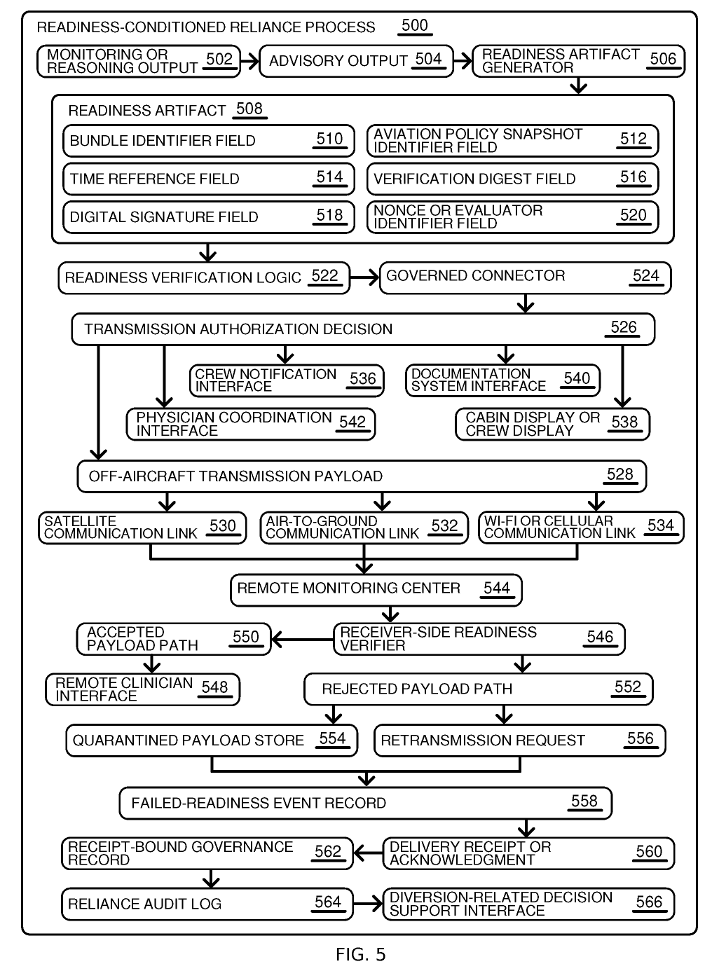

FIG. 5 - Readiness-Conditioned Reliance and Remote Monitoring

FIG. 5 illustrates a readiness-conditioned reliance process 500 configured to control whether an output generated from aircraft-cabin medical telemetry may be transmitted, displayed, documented, forwarded, accepted by a remote monitoring center, presented to a clinician, or otherwise used in a downstream reliance event.

A monitoring or reasoning output 502 may be generated by a monitoring or reasoning component after execution through a constrained execution interface using a governed context bundle. The monitoring or reasoning output 502 may include a signal interpretation, alert, recommendation, trend, risk indication, status summary, remote monitoring summary, documentation-ready record, or other output derived from patient-scoped aircraft-cabin telemetry.

An advisory output 504 may be generated for cabin crew, onboard medical personnel, a remote monitoring center, or another authorized user or system. The advisory output 504 may include crew-facing guidance, recommended next steps, telemetry summary, source-quality indication, confidence indication, escalation state, transmission state, or readiness state. The advisory output 504 does not automatically command an aircraft operation or medical intervention.

A readiness artifact generator 506 generates a readiness artifact 508. In some embodiments, the readiness artifact generator 506 may operate after governed context bundle assembly, execution governance artifact verification, monitoring or reasoning execution, or output generation. In other embodiments, the readiness artifact generator 506 may also verify an existing readiness artifact received from another authorized component. The readiness artifact 508 provides a machine-verifiable indication that a downstream reliance event is permitted under the applicable governance conditions.

The readiness artifact 508 may include a bundle identifier field 510. The bundle identifier field 510 identifies the governed context bundle used to generate the monitoring or reasoning output 502. The readiness artifact 508 may also include an aviation policy snapshot identifier field 512. The aviation policy snapshot identifier field 512 identifies the aviation policy snapshot under which the underlying memory atoms were admitted, transformed, denied, or otherwise evaluated.

The readiness artifact 508 may include a time reference field 514. The time reference field 514 may include a generation time, verification time, expiration time, task time, flight time reference, or another time value used to determine whether the readiness artifact 508 is current. The readiness artifact 508 may include a verification digest field 516. The verification digest field 516 may be computed over at least one of the governed context bundles, execution governance artifact, monitoring output, policy snapshot identifier, task identifier, or selected readiness fields.

A digital signature field 518 may provide cryptographic verification of the readiness artifact 508. The digital signature field 518 may be generated by a cabin gateway, clinical integration plane, governance service, remote monitoring service, trusted execution environment, policy server, or other authorized component. A nonce or evaluator identifier field 520 may identify a verification instance, issuing component, evaluator, policy service, or other source of the readiness artifact 508.

Readiness verification logic 522 evaluates the readiness artifact 508 before a downstream reliance event is permitted. The readiness verification logic 522 may check whether the readiness artifact 508 is present, signed, unexpired, bound to the correct governed context bundle, bound to the correct aviation policy snapshot, consistent with the execution governance artifact, consistent with the monitoring or reasoning output 502, and valid for the requested downstream reliance event. If the readiness artifact 508 fails verification, the downstream reliance event may be denied, deferred, quarantined, limited, or prevented.

A governed connector 524 receives the monitoring or reasoning output 502 and the readiness artifact 508. The governed connector 524 controls whether the output may be transmitted, displayed, documented, forwarded, or otherwise used. The governed connector 524 may serve as a controlled egress path for aircraft-cabin medical telemetry, alerts, recommendations, summaries, or other patient-scoped outputs.

A transmission authorization decision 526 may be generated by the governed connector 524 or associated readiness verification logic 522. The transmission authorization decision 526 may indicate that transmission is allowed, denied, deferred, limited, quarantined, or permitted only in a non-actionable form. The transmission authorization decision 526 may be based on readiness artifact verification, aviation policy snapshot state, consent state, remote monitoring availability, communication state, route or jurisdictional rule, payload type, crew role, or emergency-mode status.

When transmission is permitted, an off-aircraft transmission payload 528 may be generated. The off-aircraft transmission payload 528 may include telemetry, alerts, recommendations, summaries, device status, identity confidence state, governed context identifiers, readiness artifact fields, execution governance artifact references, audit references, or a limited subset of such information. The off-aircraft transmission payload 528 may be minimized, encrypted, signed, compressed, redacted, abstracted, or otherwise transformed according to policy.

The off-aircraft transmission payload 528 may be transmitted through a satellite communication link 530, an air-to-ground communication link 532, a Wi-Fi or cellular communication link 534, or another available communication path. The governed connector 524 may select or restrict the communication path based on availability, policy, route, jurisdiction, bandwidth, latency, security state, cost, priority, or emergency status.

The readiness-conditioned reliance process 500 may also control local reliance. A crew notification interface 536 may receive an advisory, alert, readiness state, failed-readiness indication, or request for additional action. A cabin display or crew display 538 may present permitted information to cabin crew or onboard medical personnel. A documentation system interface 540 may receive only information permitted for documentation under the applicable readiness state and aviation policy snapshot. A physician coordination interface 542 may be used to coordinate with a remote physician, onboard clinician, ground medical support service, or operator medical desk.

A remote monitoring center 544 may receive the off-aircraft transmission payload 528. The remote monitoring center 544 may be operated by an airline, medical support service, hospital, telemedicine provider, emergency response center, or other authorized entity. Before the received payload is accepted, stored, displayed, forwarded, documented, or acted upon, a receiver-side readiness verifier 546 may verify the readiness artifact 508. The receiver-side readiness verifier 546 may check the bundle identifier field 510, aviation policy snapshot identifier field 512, time reference field 514, verification digest field 516, digital signature field 518, nonce or evaluator identifier field 520, and any associated execution governance artifact or policy record.

A remote clinician interface 548 may present telemetry, alerts, recommendations, summaries, or advisory information to a remote clinician only after the receiver-side readiness verifier 546 determines that the readiness artifact 508 is valid for the intended reliance. In some embodiments, the remote clinician interface 548 may also display source-quality information, identity confidence state, policy state, readiness state, telemetry time window, device source, and transformation history.

An accepted payload path 550 may be used when the receiver-side readiness verifier 546 confirms that the payload is valid for the requested reliance event. The accepted payload path 550 may allow storage, display, documentation, forwarding, physician review, crew coordination, or other permitted downstream use. A rejected payload path 552 may be used when verification fails. The rejected payload path 552 may prevent the payload from being presented as actionable medical information.

A quarantined payload store 554 may hold a payload that cannot yet be accepted but should not be discarded. Quarantine may occur when a readiness artifact is missing, expired, mismatched, unsigned, inconsistent with the payload, inconsistent with the governed context bundle, inconsistent with the aviation policy snapshot, or pending later confirmation. A retransmission request 556 may be generated when the remote monitoring center 544 requires a corrected readiness artifact, corrected payload, updated policy identifier, updated bundle identifier, or retransmission after a communication failure.

A failed-readiness event record 558 may be generated when the readiness artifact 508 fails verification or when a payload is rejected, quarantined, or prevented from being used as actionable medical information. The failed-readiness event record 558 may include the reason for failure, payload identifier, readiness artifact identifier, bundle identifier, policy snapshot identifier, time of failure, receiving system identifier, and requested corrective action.

A delivery receipt or acknowledgment 560 may be returned after delivery, acceptance, rejection, quarantine, retransmission request, or readiness verification. The delivery receipt or acknowledgment 560 may be generated by the remote monitoring center 544, governed connector 524, communication link, documentation system, crew interface, or other downstream system.

A receipt-bound governance record 562 may be stored in response to the delivery receipt or acknowledgment 560. The receipt-bound governance record 562 may associate the transmission authorization decision 526, off-aircraft transmission payload 528, readiness artifact 508, remote monitoring center 544, receiver-side verification outcome, delivery status, acceptance state, rejection state, quarantine state, or failed-readiness event record 558. The receipt-bound governance record 562 may be used to prove whether a downstream reliance event was permitted, blocked, accepted, or rejected.

A reliance audit log 564 may store records of readiness artifact generation, readiness verification, transmission authorization, payload creation, local display, crew notification, documentation, remote monitoring center receipt, receiver-side verification, acceptance, rejection, quarantine, retransmission request, and clinical presentation. The reliance audit log 564 may support reconstruction of the path from monitoring output to downstream reliance.

A diversion-related decision support interface 566 may receive only information that satisfies the applicable readiness verification and policy conditions. The diversion-related decision support interface 566 does not autonomously command an aircraft diversion. Instead, it may provide permitted medical status, telemetry summary, remote clinician input, readiness state, or advisory information to crew, dispatch, or other authorized personnel while preserving crew authority and operator procedures.

Accordingly, FIG. 5 shows that a monitoring or reasoning output 502 is not automatically relied upon merely because it was generated. The output must pass through readiness artifact verification, governed connector control, and, when transmitted, receiver-side readiness verification before it is accepted, displayed, documented, forwarded, or used in a downstream reliance event.

### REFERENCE LIST

500 — READINESS-CONDITIONED RELIANCE PROCESS 502 — MONITORING OR REASONING OUTPUT 504 — ADVISORY OUTPUT 506 — READINESS ARTIFACT GENERATOR 508 — READINESS ARTIFACT 510 — BUNDLE IDENTIFIER FIELD 512 — AVIATION POLICY SNAPSHOT IDENTIFIER FIELD 514 — TIME REFERENCE FIELD 516 — VERIFICATION DIGEST FIELD 518 — DIGITAL SIGNATURE FIELD 520 — NONCE OR EVALUATOR IDENTIFIER FIELD 522 — READINESS VERIFICATION LOGIC 524 — GOVERNED CONNECTOR 526 — TRANSMISSION AUTHORIZATION DECISION 528 — OFF-AIRCRAFT TRANSMISSION PAYLOAD 530 — SATELLITE COMMUNICATION LINK 532 — AIR-TO-GROUND COMMUNICATION LINK 534 — WI-FI OR CELLULAR COMMUNICATION LINK 536 — CREW NOTIFICATION INTERFACE 538 — CABIN DISPLAY OR CREW DISPLAY 540 — DOCUMENTATION SYSTEM INTERFACE 542 — PHYSICIAN COORDINATION INTERFACE 544 — REMOTE MONITORING CENTER 546 — RECEIVER-SIDE READINESS VERIFIER 548 — REMOTE CLINICIAN INTERFACE 550 — ACCEPTED PAYLOAD PATH 552 — REJECTED PAYLOAD PATH 554 — QUARANTINED PAYLOAD STORE 556 — RETRANSMISSION REQUEST 558 — FAILED-READINESS EVENT RECORD 560 — DELIVERY RECEIPT OR ACKNOWLEDGMENT 562 — RECEIPT-BOUND GOVERNANCE RECORD 564 — RELIANCE AUDIT LOG 566 — DIVERSION-RELATED DECISION SUPPORT INTERFACE

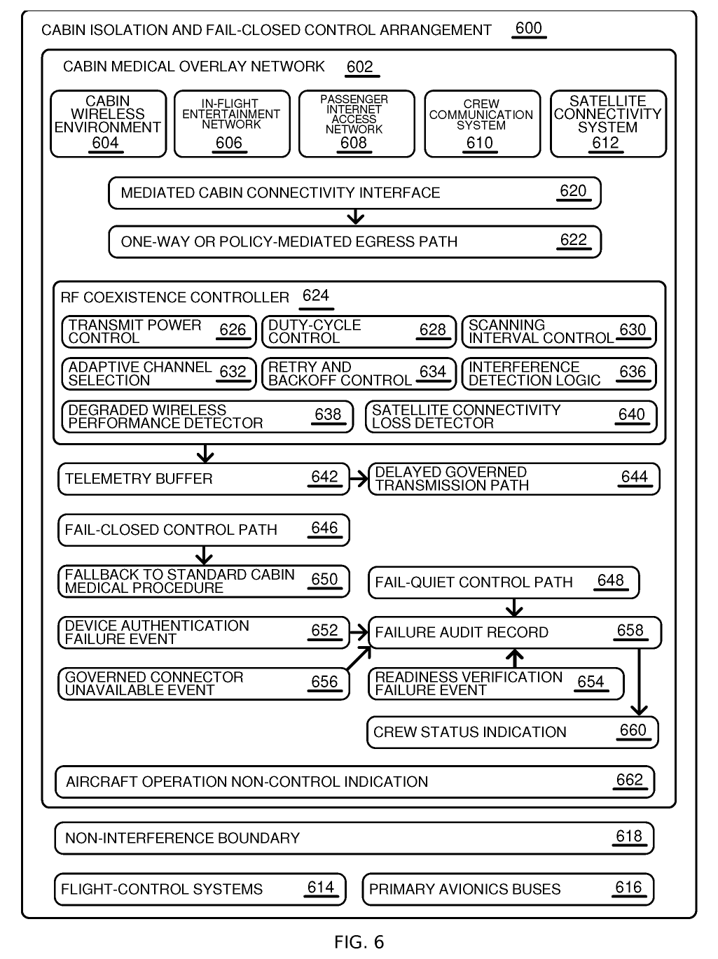

FIG. 6 - Cabin Isolation, RF Coexistence, and Fail-Closed Operation

FIG. 6 illustrates a cabin isolation and fail-closed control arrangement 600 configured to allow aircraft-cabin medical telemetry capture and governed transmission while maintaining non-interference with aircraft operational systems and adapting to cabin communication constraints.

The cabin isolation and fail-closed control arrangement 600 includes a cabin medical overlay network 602. The cabin medical overlay network 602 provides a controlled communication and processing environment for telemetry received from wearable devices, portable medical devices, medical-grade devices, proxy-capable cabin nodes, crew devices, passenger devices, dongles, adapters, gateways, or other cabin components. The cabin medical overlay network 602 may be implemented as a retrofit overlay, portable emergency-use overlay, software-defined overlay, logically isolated network segment, or combination of physical and logical communication paths.

The cabin medical overlay network 602 operates within a cabin wireless environment 604. The cabin wireless environment 604 may include passenger devices, crew devices, wearable devices, medical devices, seat electronics, wireless access points, cabin gateways, emergency equipment, service carts, and other radio-frequency sources present in an aircraft cabin. Because the cabin wireless environment 604 may be congested, variable, or constrained by operator procedures, the cabin medical overlay network 602 may be configured to adapt communication behavior without disrupting cabin systems or aircraft operations.

The cabin isolation and fail-closed control arrangement 600 may coexist with an in-flight entertainment network 606. The in-flight entertainment network 606 may provide passenger entertainment, seat-back display communication, media distribution, passenger services, or related cabin functions. The cabin medical overlay network 602 may be logically separated from the in-flight entertainment network 606 so that medical telemetry processing does not require modification of entertainment infrastructure and so that entertainment data is not treated as governed medical telemetry.

The arrangement 600 may also coexist with a passenger Internet access network 608. The passenger Internet access network 608 may provide general passenger connectivity. In some embodiments, the cabin medical overlay network 602 does not use the passenger Internet access network 608 except through a mediated or policy-controlled interface. When passenger connectivity is used as a transport path, telemetry, summaries, readiness artifacts, or other medical payloads may remain subject to encryption, readiness verification, policy-mediated egress, and audit logging.

A crew communication system 610 may be present in or associated with the aircraft cabin. The crew communication system 610 may provide crew messaging, crew coordination, operator communication, or emergency communication. The cabin medical overlay network 602 may provide crew-facing medical status or readiness-state information through an authorized interface without assuming control of crew communication functions.

A satellite connectivity system 612 may provide off-aircraft communication. The satellite connectivity system 612 may be used by the governed connector for remote monitoring transmission when readiness verification and policy conditions are satisfied. In some embodiments, the satellite connectivity system 612 is unavailable, bandwidth-limited, delayed, intermittent, or restricted by flight segment, route, operator policy, or aircraft state. The cabin medical overlay network 602 may therefore buffer telemetry, defer transmission, send a reduced payload, or fail closed when governed transmission cannot be completed.

The arrangement 600 is separated from flight-control systems 614 and primary avionics buses 616. The flight-control systems 614 may include systems involved in aircraft navigation, propulsion control, flight-control surfaces, autopilot control, flight management, or other aircraft operational control. The primary avionics buses 616 may include aircraft data buses or avionics communication paths used for flight operations. The cabin medical overlay network 602 is not configured to command, alter, or interfere with the flight-control systems 614 or the primary avionics buses 616.

A non-interference boundary 618 separates the cabin medical overlay network 602 from the flight-control systems 614 and primary avionics buses 616. The non-interference boundary 618 may be implemented by physical separation, network segmentation, firewall rules, one-way communication rules, absence of avionics interfaces, policy restrictions, hardware isolation, software access controls, certification boundaries, or operational procedures. In some embodiments, the non-interference boundary 618 indicates that the system may provide advisory medical information but does not issue commands to aircraft navigation, propulsion, flight-control, or primary avionics functions.

A mediated cabin connectivity interface 620 may connect the cabin medical overlay network 602 to permitted cabin communication resources. The mediated cabin connectivity interface 620 may be used to access cabin Wi-Fi, crew communication infrastructure, satellite connectivity, air-to-ground connectivity, cellular connectivity, or another permitted communication path. The mediated cabin connectivity interface 620 may enforce authentication, authorization, encryption, payload filtering, readiness verification, policy rules, and audit logging before data is transmitted outside the cabin medical overlay network 602.

A one-way or policy-mediated egress path 622 may be used for off-aircraft transmission or downstream system communication. The one-way or policy-mediated egress path 622 may allow governed telemetry, alerts, recommendations, summaries, readiness artifacts, or audit records to leave the cabin medical overlay network 602 only when conditions are satisfied. The path 622 may prevent uncontrolled inbound commands, prevent ungoverned patient-scoped data egress, or restrict communication to approved payload types and destinations.

An RF coexistence controller 624 manages communication behavior within the cabin wireless environment 604. The RF coexistence controller 624 may monitor communication quality, interference, channel occupancy, connection reliability, cabin network availability, device density, or operator-defined RF constraints. The RF coexistence controller 624 may adjust radio behavior to reduce interference, preserve telemetry quality, and avoid unnecessary radio transmissions.

The RF coexistence controller 624 may apply transmit power control 626. Transmit power control 626 may reduce, increase, cap, or otherwise regulate transmission power of proxy-capable cabin nodes, dongles, gateways, crew devices, passenger devices, or medical telemetry radios. The RF coexistence controller 624 may also apply duty-cycle control 628. Duty-cycle control 628 may limit how frequently a node transmits, scans, advertises, retries, or forwards telemetry.

Scanning interval control 630 may adjust scan frequency for detecting wearable devices, medical devices, proxy nodes, or communication links. Adaptive channel selection 632 may select among available communication channels based on interference, cabin network state, link quality, policy, device type, or communication priority. Retry and backoff control 634 may determine when and how often a node attempts retransmission after packet loss, link failure, gateway unavailability, or degraded wireless performance.

Interference detection logic 636 may detect radio interference, excessive packet loss, channel congestion, unexpected signal conditions, communication collisions, or coexistence conflicts with cabin systems. A degraded wireless performance detector 638 may identify loss of link quality, low signal strength, increasing latency, excessive retries, incomplete telemetry frames, unstable proxy connections, or other communication degradation. A satellite connectivity loss detector 640 may determine when off-aircraft connectivity is unavailable, degraded, restricted, or insufficient for governed transmission.

A telemetry buffer 642 stores telemetry, normalized telemetry objects, memory atoms, transmission payloads, readiness artifacts, audit records, or other records when immediate forwarding is not permitted or not possible. The telemetry buffer 642 may be located in a proxy-capable cabin node, crew device, passenger device, dongle, gateway, clinical integration plane, or other edge component. Buffered records may be marked with event time, receipt time, sequence information, freshness state, policy state, and transmission state.

A delayed governed transmission path 644 may be used when telemetry or a derived payload cannot be transmitted immediately. The delayed governed transmission path 644 may send a payload after connectivity is restored, after readiness verification is completed, after identity or consent is confirmed, after a policy state is updated, or after a remote monitoring center becomes available. In some embodiments, delayed transmission is allowed only if freshness, ordering, consent, policy, and readiness conditions remain satisfied.

A fail-closed control path 646 may be invoked when the system cannot satisfy a required governance, readiness, authentication, connectivity, or policy condition. Under the fail-closed control path 646, the system may deny transmission, deny patient-specific execution, prevent downstream reliance, block display as actionable information, prevent documentation as verified clinical information, or quarantine a payload. The fail-closed control path 646 may preserve captured records for audit while preventing unauthorized reliance.

A fail-quiet control path 648 may be invoked when continued transmission or alerting may create interference, confusion, alarm fatigue, or unreliable output. Under the fail-quiet control path 648, the system may reduce transmission frequency, stop nonessential scanning, suspend noncritical alerts, suppress repeated failed transmissions, or provide a limited status indication rather than continuing to generate uncertain outputs. The fail-quiet control path 648 may be used when RF conditions degrade, satellite connectivity fails, readiness verification fails, or device authentication cannot be established.

A fallback to standard cabin medical procedure 650 may occur when the aircraft-cabin medical telemetry system cannot provide governed telemetry or readiness-verified outputs. The fallback 650 may instruct crew to follow existing emergency procedures, use onboard medical kits, consult remote medical support by voice when available, obtain manual measurements, request assistance from onboard medical personnel, or continue other operator-approved procedures. The fallback 650 preserves crew authority and does not prevent standard emergency care.

A device authentication failure event 652 may occur when a wearable device, medical device, dongle, adapter, proxy node, crew device, passenger device, or gateway cannot be authenticated or authorized. In response, telemetry from the device or node may be denied, quarantined, assigned lower confidence, stored for audit only, or excluded from a governed context bundle. A readiness verification failure event 654 may occur when a readiness artifact is absent, invalid, expired, unsigned, mismatched, or inconsistent with a governed context bundle or aviation policy snapshot. In response, the system may deny, defer, quarantine, or limit the downstream reliance event.

A governed connector unavailable event 656 may occur when the governed connector cannot transmit, verify readiness, reach an approved destination, access the permitted communication path, or obtain a required acknowledgment. In response, the system may buffer the payload, create a failure record, provide crew status, attempt delayed governed transmission, or fail closed according to policy.

A failure audit record 658 may be generated for a device authentication failure event 652, readiness verification failure event 654, governed connector unavailable event 656, RF interference condition, degraded wireless condition, satellite connectivity loss, fail-closed action, fail-quiet action, fallback event, or delayed transmission. The failure audit record 658 may include time, source, affected passenger partition, device identifier, proxy node identifier, policy snapshot identifier, readiness artifact identifier, attempted action, failure reason, and resulting system response.

A crew status indication 660 may be presented to cabin crew through a crew interface, crew display, mobile device, or other authorized surface. The crew status indication 660 may indicate that telemetry capture is active, transmission is delayed, readiness verification failed, a device is unauthenticated, a payload is quarantined, the system is operating in fail-quiet mode, or standard cabin medical procedures should be followed. The crew status indication 660 may be limited to information permitted by policy and crew role.

An aircraft operation non-control indication 662 may be stored, displayed, or represented by system configuration to confirm that the aircraft-cabin medical telemetry system provides advisory medical telemetry handling and does not command aircraft navigation, propulsion, flight-control, or primary avionics functions. The indication 662 may be reflected in system architecture, operational manuals, configuration records, audit records, crew-facing status, or certification documentation.

Accordingly, FIG. 6 shows that the aircraft-cabin medical telemetry architecture is not merely a wireless telemetry system placed in an aircraft. The architecture includes isolation from flight-control systems and primary avionics buses, controlled use of cabin connectivity, policy-mediated egress, RF coexistence behavior, buffering, delayed governed transmission, fail-closed and fail-quiet modes, failure audit records, and fallback to standard cabin medical procedures when governed telemetry reliance is not available.

### REFERENCE LIST

600 — CABIN ISOLATION AND FAIL-CLOSED CONTROL ARRANGEMENT 602 — CABIN MEDICAL OVERLAY NETWORK 604 — CABIN WIRELESS ENVIRONMENT 606 — IN-FLIGHT ENTERTAINMENT NETWORK 608 — PASSENGER INTERNET ACCESS NETWORK 610 — CREW COMMUNICATION SYSTEM 612 — SATELLITE CONNECTIVITY SYSTEM 614 — FLIGHT-CONTROL SYSTEMS 616 — PRIMARY AVIONICS BUSES

618 — NON-INTERFERENCE BOUNDARY 620 — MEDIATED CABIN CONNECTIVITY INTERFACE 622 — ONE-WAY OR POLICY-MEDIATED EGRESS PATH 624 — RF COEXISTENCE CONTROLLER 626 — TRANSMIT POWER CONTROL 628 — DUTY-CYCLE CONTROL 630 — SCANNING INTERVAL CONTROL 632 — ADAPTIVE CHANNEL SELECTION 634 — RETRY AND BACKOFF CONTROL 636 — INTERFERENCE DETECTION LOGIC 638 — DEGRADED WIRELESS PERFORMANCE DETECTOR 640 — SATELLITE CONNECTIVITY LOSS DETECTOR 642 — TELEMETRY BUFFER 644 — DELAYED GOVERNED TRANSMISSION PATH 646 — FAIL-CLOSED CONTROL PATH 648 — FAIL-QUIET CONTROL PATH 650 — FALLBACK TO STANDARD CABIN MEDICAL PROCEDURE 652 — DEVICE AUTHENTICATION FAILURE EVENT 654 — READINESS VERIFICATION FAILURE EVENT 656 — GOVERNED CONNECTOR UNAVAILABLE EVENT 658 — FAILURE AUDIT RECORD 660 — CREW STATUS INDICATION 662 — AIRCRAFT OPERATION NON-CONTROL INDICATION

1. A system for in-flight patient monitoring in an aircraft cabin, comprising:
one or more processors; and
memory storing instructions that, when executed by the one or more processors, cause the system to:
enforce execution of a monitoring or reasoning component through a constrained execution interface that permits access to patient-scoped contextual information only upon validation of a governed execution package;
receive telemetry from at least one wearable device or medical device through at least one proxy-capable aircraft cabin node deployed within, carried in, or temporarily active within the aircraft cabin;
associate the telemetry with a passenger identity using passenger identity resolution based on at least one of a passenger manifest, seat assignment, consent state, device association, passenger mobile credential, crew verification, emergency token, temporary patient profile, or post-event reconciliation;
store the telemetry or one or more derived values as memory atoms in a patient-partitioned contextual memory associated with the passenger identity, each memory atom including telemetry or derived contextual information and associated provenance metadata;
select candidate memory atoms responsive to a monitoring task request;
evaluate, prior to execution of the monitoring or reasoning component, the candidate memory atoms using a non-bypassable memory gate under an aviation policy snapshot and one or more integrity criteria; determine, by the non-bypassable memory gate, an admissibility state for each candidate memory atom; prevent use of any denied memory atom or quarantined memory atom as patient-scoped execution input, and permit a redacted memory atom, abstracted or attenuated memory atom, or non-data-bearing transformed record to be used only according to the admissibility state determined by the non-bypassable memory gate; assemble admitted memory atoms and permitted transformed records into a governed context bundle; generate an execution governance artifact bound to the governed context bundle and the aviation policy snapshot; generate the governed execution package comprising the governed context bundle and the execution governance artifact; permit execution of the monitoring or reasoning component only when the governed execution package is presented to and validated by the constrained execution interface; reject, deny, defer, quarantine, or limit execution when the governed execution package is absent, invalid, expired, or inconsistent with the aviation policy snapshot; and condition a downstream reliance event on verification of a readiness artifact bound to the governed context bundle and the aviation policy snapshot.

2. The system of claim 1, wherein the proxy-capable aircraft cabin node comprises at least one of a seat-integrated wireless endpoint, crew-carried mobile device, passenger mobile device executing a proxy application, portable medical dongle, medical device adapter, cabin gateway, cart-mounted device, wall-powered node, battery-powered node, or temporarily active emergency node.

3. The system of claim 1, wherein the provenance metadata includes at least one of a device identifier, device type, proxy node identifier, seat identifier, cabin location identifier, flight identifier, passenger identity reference, consent state, timestamp, time-validity interval, source trust classification, integrity status, route or jurisdiction indicator, transformation history, or provenance chain.

4. The system of claim 1, wherein the aviation policy snapshot encodes at least one of airline operator rules, route-dependent requirements, jurisdictional privacy requirements, consent rules, credentialing constraints, escalation thresholds, retention rules, crew role permissions, remote clinician permissions, satellite connectivity constraints, emergency-mode overrides, or flight-phase-dependent rules.

5. The system of claim 1, wherein the aviation policy snapshot is updated responsive to at least one of route, jurisdiction, flight phase, connectivity state, passenger consent state, crew role, escalation status, remote monitoring availability, or emergency-mode activation, and wherein the governed context bundle or readiness artifact is bound to an identifier or digest of the aviation policy snapshot.

6. The system of claim 1, wherein the non-bypassable memory gate assigns to each candidate memory atom an admissibility state comprising admission, denial, redaction, abstraction, attenuation, quarantine, or transformation into a non-data-bearing form.

7. The system of claim 1, wherein the one or more integrity criteria comprise at least one of provenance verification, temporal validity evaluation, source trust classification, conflict detection, completeness evaluation, device-authentication status, seat-identity consistency, consent status, route-policy status, or transformation-history evaluation.

8. The system of claim 1, wherein the governed context bundle comprises at least one of an included set of admitted memory atoms and permitted transformed records, a bundle identifier, a bundle digest, a policy snapshot identifier, a timestamp, a passenger identity reference, a flight identifier, a task identifier, or a record of transformations applied by the non-bypassable memory gate.

9. The system of claim 1, wherein the monitoring or reasoning component is prevented from accessing patient-scoped contextual information outside the governed context bundle, and wherein execution is denied, delayed, quarantined, or limited to non-patient-specific fallback content when the governed context bundle or execution governance artifact is absent, invalid, expired, mismatched, or inconsistent with the aviation policy snapshot.

10. The system of claim 1, wherein the readiness artifact comprises at least one of a bundle identifier, aviation policy snapshot identifier, time reference, verification digest, digital signature, nonce, evaluator identifier, execution certificate, readiness certificate, policy-verification result, or transmission authorization record.

11. The system of claim 1, wherein a governed connector conditions satellite, air-to-ground, Wi-Fi, cellular, or other off-aircraft transmission of telemetry, alerts, recommendations, or summaries to a remote monitoring center on successful verification of the readiness artifact.

12. The system of claim 1, wherein the system operates as a cabin medical overlay network logically isolated from flight-control systems and primary avionics buses and is configured so that medical telemetry processing does not command, alter, or interfere with aircraft navigation, propulsion, flight-control, or primary avionics functions.

13. A computer-implemented method of in-flight patient monitoring in an aircraft cabin, comprising:
receiving telemetry from at least one wearable device or medical device through at least one proxy-capable aircraft cabin node;
resolving or associating a passenger identity using at least one of manifest information, seat assignment, consent state, device association, passenger mobile credential, crew verification, emergency token, temporary patient profile, or post-event reconciliation;
storing the telemetry or one or more derived values as memory atoms in a patient-partitioned contextual memory associated with the passenger identity;
selecting candidate memory atoms responsive to a monitoring task request;
evaluating the candidate memory atoms under an aviation policy snapshot using a non-bypassable memory gate before execution of a monitoring or reasoning component;
assembling admitted memory atoms and permitted transformed records into a governed context bundle;
executing the monitoring or reasoning component only through a constrained execution interface using the governed context bundle and an execution governance artifact; and
conditioning a downstream reliance event on verification of a readiness artifact bound to the governed context bundle and the aviation policy snapshot.

14. The method of claim 13, further comprising denying, deferring, quarantining, limiting, or preventing at least one of crew notification, off-aircraft transmission, rendering, storage as actionable medical information, forwarding, clinical display, remote monitoring center acceptance, physician coordination, documentation, or diversion-related decision support when the readiness artifact is absent, invalid, expired, unsigned, mismatched, or inconsistent with the governed context bundle or aviation policy snapshot.

15. The method of claim 13, further comprising performing proxy handoff by selecting a different proxy-capable aircraft cabin node responsive to passenger movement, node unavailability, link-quality degradation, battery state, connectivity state, authentication state, or cabin-location change while preserving provenance continuity between a prior proxy node and a newly selected proxy node.

16. The method of claim 13, further comprising maintaining separate flight-session memory regions for multiple passengers within the same aircraft cabin, independently evaluating candidate memory atoms for different passenger identities, and generating distinct governed context bundles, readiness artifacts, and audit records for the different passenger identities.

17. A non-transitory computer-readable medium storing instructions that, when executed by one or more processors, cause the one or more processors to perform operations comprising:
receiving aircraft-cabin medical telemetry through at least one proxy-capable aircraft cabin node;
associating the aircraft-cabin medical telemetry with a passenger identity and storing the aircraft-cabin medical telemetry or derived values as memory atoms in a patient-partitioned contextual memory;
selecting candidate memory atoms responsive to a monitoring task request;
evaluating the candidate memory atoms under an aviation policy snapshot using a non-bypassable memory gate before execution of a monitoring or reasoning component;
assembling admitted memory atoms and permitted transformed records into a governed context bundle;
permitting the monitoring or reasoning component to execute only through a constrained execution interface using the governed context bundle and an execution governance artifact;
generating or verifying a readiness artifact bound to the governed context bundle and the aviation policy snapshot; and
conditioning a downstream reliance event on verification of the readiness artifact.

18. The non-transitory computer-readable medium of claim 17, wherein the operations further comprise transmitting telemetry, alerts, recommendations, or summaries to a remote monitoring center through a governed connector only after successful verification of the readiness artifact, and wherein the remote monitoring center verifies the readiness artifact before accepting, storing, displaying, forwarding, documenting, or acting upon the telemetry, alerts, recommendations, or summaries.

19. The non-transitory computer-readable medium of claim 17, wherein the operations further comprise storing a receipt-bound governance record responsive to at least one of a delivery receipt, acknowledgment, delivery status indication, readiness verification outcome, receiver-side quarantine event, failed-readiness event, or remote monitoring center verification result.

20. The non-transitory computer-readable medium of claim 17, wherein the operations further comprise adapting at least one of transmit power, duty cycle, scanning interval, channel selection, retry behavior, buffering behavior, proxy selection, or fail-quiet operation responsive to cabin radio coexistence constraints, degraded wireless performance, satellite connectivity loss, or detected interference.
